(19) United States
(12) Patent Application Publication
(10) Pub. No.: US 2014/0322340 A1
(43) Pub. Date: Oct. 30, 2014

(54) PROTEIN-POLYSACCHARIDE CONJUGATES AND USE FOR ENCAPSULATING NUTRACEUTICALS FOR CLEAR BEVERAGE APPLICATIONS

(71) Applicant: Technion Research and Development Foundation LTD., Haifa (IL)

(72) Inventor: Yoav D. Livney, Atzmon-Segev (IL)

(73) Assignee: Technion Research and Development Foundation LTD., Haifa (IL)

(21) Appl. No.: 14/269,153

(22) Filed: May 4, 2014

Related U.S. Application Data
(63) Continuation of application No. 13/410,001, filed on Mar. 1, 2012, now abandoned.
(60) Provisional application No. 61/447,773, filed on Mar. 1, 2011.

(51) Int. Cl. A61K 9/51; A61K 31/355; A23L 2/52; A61K 31/352; A61K 31/536; A61K 31/201; A61K 31/07; A61K 31/59

(52) U.S. Cl. CPC A61K 9/5169; A61K 31/07; A61K 31/355; A61K 31/59; A61K 31/352; A61K 31/536; A61K 31/201; A23L 2/52; B82Y 5/00
USPC 424/491; 424/400; 514/725; 514/458; 514/167; 514/456; 514/229.8; 514/560; 977/773; 977/915

(57) ABSTRACT

The present invention provides protein (or peptide)-polysaccharide (or oligosaccharide) conjugates (PPC) as nanocapsular vehicles for nanoencapsulation of biologically active compounds, particularly nutraceuticals. The PPCs efficiently protect both hydrophobic (i.e., water insoluble) and hydrophilic (i.e., water soluble) nutraceuticals, to provide a composition which, when added to a beverage, disperses so as to provide a clear or transparent solution. In some embodiments, the PPCs are Maillard reaction based PPCs. Advantageously, the conjugates of the present invention protect the nutraceuticals from degradation, both during shelf life and upon gastric digestion.

### RELATED APPLICATIONS

This application claims the benefit of and priority from U.S. Provisional Application No. 61/447,773, filed on Mar. 1, 2011, the entirety of which is incorporated herein by reference for the teachings therein.

### FIELD OF THE INVENTION

The present invention relates to the field of food technology and delivery of biologically active compounds via beverages and food. In particular the present invention provides protein (or peptide)-polysaccharide (or oligosaccharide) conjugates, and use thereof for encapsulation, stabilization and protection of active compounds, in particular for clear beverage applications.

### BACKGROUND OF THE INVENTION

One of the most important aims of contemporary food engineering is the enrichment of foods with health enhancing components. There is a growing public awareness for healthy nourishment that includes daily amounts of required micronutrients such as vitamins, essential fatty acids and antioxidants. Along with the tendency to enrich foods and drinks with healthy compounds there is a trend to exclude potentially harmful compounds.

A sub-category of healthy food is nutraceuticals-enriched food, in which a health-promoting bioactive molecule is added to the food or beverage. Many of the nutraceuticals which are desired for enrichment of food and beverages are hydrophobic (hydrophobic nutraceuticals or HN) and thus poorly water soluble, or water-insoluble. Some examples are vitamin A, vitamin D, vitamin E, carotenoids, and ω-3 fatty acids. Many of these HN are also sensitive to oxidation, and other degradation mechanisms.

Enrichment of food and beverages with sensitive HN is a challenge for several reasons: (a) the poor solubility of HN in water, which necessitates the use of a surface active agent (surfactant); (b) the surfactant-HN nanovehicle, or nanocapsule, must be colloidally stable in the target product environment (e.g., temp, pH, ionic strength) during the production and shelf life of the product; (c) HN-loaded nanoparticles must be as small as possible in order to minimize the effect on turbidity (visible light scattering); (d) if the HN is sensitive to oxidation, the vehicle should confer protection to retard HN degradation during shelf life. Oxidation reactions can be retarded by either antioxidant activity of substances in the environment or by encapsulating the sensitive material preventing heavy metals and oxygen from nearing the sensitive HN, and reducing their mobility and reactivity; and (e) all materials composing the vehicle and procedures of its formation must be defined as “generally recognized as safe” (GRAS). Natural food materials and common procedures in food processing are thus preferred.

Several methods have been introduced for enrichment of HN in aqueous solutions, mainly (a) emulsions stabilized by synthetic surfactants such as polysorbate (Tween); (b) emulsions stabilized by low MW natural surfactants such as phospholipids or monoglycerides; and (c) proteins as emulsifiers and nanocapsules (e.g., casein). WO 2007/122613 to the inventor of the present invention describes a system based on re-assembled casein micelles for the delivery of hydrophobic biologically active compounds in food and beverages. US patent application No. US 2011/038987 to the inventor of the present invention teaches the use of beta casein assemblies for enrichment of food and beverages, however the stability of beta casein around pH 4.5-5.5 is very poor, and the protection it provides to sensitive HN is limited. US patent application No. US 2011/0038942 to the inventor of the present invention teaches the formation of beta-lactoglobulin-polysaccharide nanocomplexes for hydrophobic bioactive compounds, for clear drink enrichment with HN, however the non-covalent nature of these complexes limits their application ranges in terms of pH and ionic strength.

Covalently bonded protein-polysaccharides conjugates (PPC) can also act as good emulsifier and for stabilizing agents of HN. One of the dispersant materials frequently utilized is Gum Arabic (gum acacia) which is a natural PPC composed of the polysaccharide arabinogalactan and about 2% protein. The protein regions of the gum Arabic apparently adsorb to hydrophobic droplets in solution. Gum Arabic is exudated as resin from stems and branches of acacia trees, and exhibits wide diversity in structure and properties, depending on period of year harvested, the tree's age and species. Its main disadvantages are its high price and highly variable composition and quality. Therefore, many efforts are aimed at finding good and inexpensive substitutes.^{1,2}

There have been several recent attempts to produce alternative PPC systems under controllable conditions. Possible methods for preparing protein polysaccharide linkage are by enzymatic,³ chemical⁴ or by electrosynthesis^{5,6} reactions. A particularly attractive way to form PPC is via the Maillard reaction⁷ achieved only by heating, which is typical of cooking and food processing. This is most desirable for food applications as it enables the label-friendly statement: “All natural ingredients”. In the Maillard reaction the amino groups originating from the ε-lysine or the amino terminal of the protein are conjugated to an aldehyde group of the saccharide. There are several studies reported regarding glyco-conjugation of milk proteins—whey proteins⁸⁻¹⁰ or caseins.¹¹⁻¹³ Additional substrates used for the Maillard reaction are soy proteins.¹⁴⁻¹⁶ Improvement of functional properties via Maillard conjugation of hydrolyzed soy¹⁷ and gluten¹⁸ proteins was also described.

As a result of the Maillard conjugation a “block-copolymer” with greatly improved functional properties can be obtained. Among the reported improvements are enhanced protein thermal stability, reduction of aggregate sizes,¹⁹ reduction of antigenicity,²⁰ improved solubility and antioxidant activity.^{21,22} Special attention was paid to increased emulsifying capabilities of the Maillard products. Stability tests of oil in water emulsion formation and stabilization demonstrated superiority of the conjugates over non-conjugated proteins.

Only a few examples of the use of Maillard PPCs as encapsulation materials are known in the art. For example, the micellization properties of casein-dextran grafts were studied^{23,24} and micelles of around 80 nm were produced around the pI of casein. In addition, beta-carotene encapsulation by casein-dextran grafts resulting in 200 nm core shell particles was shown.²⁵ Submicron particles of whey protein-MD conjugates were prepared for conjugated linoleic acid (CLA) encapsulation by dry heating.²⁶ The particle size range disclosed in the art is too high if transparent food solutions (e.g. clear beverages) are to be enriched, and the creation of smaller particles is desirable.

Li²⁷ used Bovine Serum Albumin (BSA) conjugated with dextran to encapsulate ibuprofen, reporting an average size of less than 100 nm, but this system was not proposed for HN delivery in transparent beverage systems, nor were any absorbance or visual results reported to support such applications. Wooster and Augustin²⁸ used maltose or MD of several sizes, conjugated to beta-Lactoglobulin as shell material for encapsulation. They used latex particles as hydrophobic core material. The conjugate-latex particles were up to about 100 nm in diameter. Hiller and Lorenzen²⁹ examined the hydrophobicity of several carbohydrates (dextran, glucose, lactose, pectin) conjugated with several proteins (casein, whey proteins, and combinations of isolated milk proteins), and have shown a decrease in surface hydrophobicity as a function of heating time even after 4 hrs of heating.

Serfert et al.³⁰ used several carbohydrates (glucose, glucose syrup, dextran) conjugated to caseins for microencapsulation of fish oil. They showed an increase in redox index after conjugation with all sugars, meaning the Maillard reaction increased the potential of the protein to act as antioxidant. O'Regan and Mulvihill³¹ used casein-MD (CN-MD) conjugates and their hydrolyzates as emulsifiers. They showed that conjugation of MD to casein increases the casein's solubility at its pI (pH=4.6).

PPCs have been studied as potential nano capsules but up to date, no method for nano encapsulation of HN was reported to form particles small enough such that, when mixed with a liquid, a clear and transparent solution is obtained.

### SUMMARY OF THE INVENTION

The present invention provides covalently bonded protein-polysaccharide conjugates (PPC) (including conjugates comprising oligosaccharides and/or peptides) as vehicles for nanoencapsulation of biologically active compounds, particularly nutraceuticals. The PPCs efficiently entrap and protect both hydrophobic (i.e., water insoluble or poorly water-soluble) and certain hydrophilic (i.e., water soluble) nutraceuticals, to provide a composition which, when added to a beverage, disperses so as to provide a clear solution. Advantageously, the conjugates of the present invention protect the nutraceuticals from degradation over a wide range of pH values, both during shelf life and upon gastric digestion. In one embodiment, the PPCs are formed by a Maillard reaction. The PPCs may comprise a Schiff base or Amadori rearrangement products, or keto-enol tautomers. In other embodiments the PPC comprises any other covalent link between the protein (or peptide) and the polysaccharide (or oligosaccharide).

The present invention departs from the known functions of PPCs (e.g., Maillard reaction-based PPCs) as a vehicle for encapsulating nutraceuticals in that it provides for nanoencapsulation of nutraceuticals for clear drink applications at high encapsulation efficiency (possibly >90%), good solubilization, stabilization and protection conferred to sensitive bioactive compounds against degradation. In contrast to known Maillard reaction-based PPC encapsulation products, which have a particle size range that is too large for formation of clear liquid solution, the compositions of the present invention can be added to beverage solution while maintaining transparency and avoiding the formation of turbid solutions or precipitation products. The nano-capsules disclosed by the invention can be incorporated into almost any beverage product without adversely modifying its properties. Advantageously, the compositions of the invention comprises only natural, generally regarded as safe (GRAS), non-toxic ingredients. As such, the compositions of the invention offer significant advantages over the prior art.

A major unique aspect of this invention is the harnessing of covalently linked conjugates of a protein (or peptide) and a polysaccharide (or oligosaccharide) for the stabilization, delivery and protection of insoluble/hydrophobic or soluble/hydrophilic biologically active compounds, particularly nutraceuticals, while maintaining the particle size of the compositions sufficiently small such that, when added to a beverage, a clear solution is formed. The encapsulated compositions not only are the ideal vehicles for stabilizing and delivering biologically active compounds, but their properties enable their incorporation into beverage products (e.g.; water, enriched and/or flavored water, sports drinks, sodas, milk, juice etc.) without compromising the properties of the solution. Furthermore, the encapsulated compositions protect the nutraceutical from degradation over a wide range of pH values (e.g., a pH range of 2.0 to 10.0), both chemically (e.g., during shelf life), or in acidic conditions such as during gastric digestion.

The advantages of the present invention:

1) Smaller size achieved by the entrapment technique, and use of oligomers (peptides and maltodextrin) enabling transparent solutions. Solubility at the protein pI.

2) The protection conferred against degradation by the encapsulation, and by the antioxidant properties of the proteins, and the Maillard reaction products. This advantage is particularly significant compared to low molecular weight emulsifiers.

3) Kosher Parve (in some of the combinations proposed) where vegetable proteins are used.

4) Low allergenicity when using hydrolyzates of proteins not considered allergenic (e.g. rice protein). These advantages are significant compared to milk protein-based systems).

5) Potentially masking of undesired flavors.

6) Potential for improved bioavailability.

According to one aspect, the present invention provides a composition for enrichment of beverages, comprising a nutraceutical encapsulated or entrapped or protected by a conjugate, the conjugate comprising a protein (or peptide) covalently bonded to a polysaccharide (or oligosaccharide) (collectively designated herein “PPC”), wherein the particle size of said composition is sufficiently small such that, when added to a beverage, a clear solution is formed. In one embodiment, the PPC comprises a Schiff base, or Amadori rearrangement products, or keto-enol tautomers based linkage between the peptide and the saccharide. Such a structure may be formed by a Maillard reaction or a Maillard-type reaction.

According to another aspect, the present invention provides a composition for enrichment of beverages, comprising a nutraceutical which is encapsulated or entrapped or protected by a conjugate, the conjugate comprising a protein or polypeptide which is covalently linked to a polysaccharide or oligosaccharide (PPC), wherein the PPC is formed by a Maillard reaction or a Maillard-type reaction, and wherein the particle size of said composition is sufficiently small such that, when added to a beverage, a clear solution is formed.

In general, the compositions of the present invention can have any average particle size as long as they result in a transparent solution when mixed with a liquid. In one embodiment, the average particle diameter of said composition is between about 50 and 100 nm. Preferably, the average particle diameter of said composition is less than about 50 nm and even more preferably less than about 20 nm. Non-limiting examples of particle diameters include less than about 50 nm, less than about 40 nm, less than about 30 nm, less than about 20 nm or less than about 10 nm. Each possibility represents a separate embodiment of the present invention. The compositions can be homogenized in order to reach the desired particle size.

The solutions of the present invention typically will have an absorbance at 600 nm of below about 0.1, preferably below about 0.075, preferably below about 0.05, more preferably below about 0.02 and even more preferably below about 0.01, with each possibility representing a separate embodiment of the present invention. In one embodiment, the clear or transparent solution has an absorbance at 600 nm of less than about 0.1.

Without wishing to be bound by any particular mechanism or theory, it is contemplated that PPC encapsulation of nutraceuticals leads to the formation of smaller particle size than simple protein encapsulation. This is because conjugation of the oligosaccharide may add steric hindrance which could lead to a small packing parameter, higher curvature, and consequently inhibition of protein aggregation and formation of smaller nanoparticles. The uniqueness of the present invention is based in part on the choice of raw materials, in particular the use of oligosaccharides (e.g. maltodextrin), along with an amphiphilic peptide or protein (e.g. casein, beta-conglycinin), which when covalently bonded under controlled conditions form conjugates (e.g., Maillard reaction conjugates), whose self-assembly, and co-assembly with the nutraceuticals, result in high particle-surface curvature, and hence small nanoparticles—thus enabling the formation of clear solutions.

The molar ratio of carbohydrate to protein used to prepare the PPCs can vary, but in general ranges from about 1:1 to about 1:50 (protein to carbohydrate). Some preferred but non-limiting rations include about 1:1, 1:5, 1:10, 1:20, 1:40 or 1:80 (protein to carbohydrate). Each possibility represents a separate embodiment of the present invention.

In addition, the molar ratio of PPC to nutraceutical can vary, but in general ranges from about 1:1 to about 1:10 (in terms of protein to nutraceutical). Some preferred but non-limiting rations include about 1:1, 1:2, 1:4, 1:6, 1:8 or 1:10 (protein to nutraceutical). Each possibility represents a separate embodiment of the present invention.

In one embodiment the nutraceutical is a hydrophobic nutraceutical (HN), i.e., it generally is poorly soluble or insoluble in water. In another embodiment, however, the nutraceutical may be a hydrophilic nutraceutical, i.e., it is moderately to highly water soluble. Each possibility represents a separate embodiment of the present invention.

In some embodiments the HN is a fat-soluble vitamin. Suitable fat-soluble vitamins include, but are not limited to vitamin D (D2, D3 and their derivatives), vitamin E (α, β, γ, δ-tocopherols, or α, β, γ, δ-tocotrienols), vitamin A (retinol, retinal, retinoic acid), and vitamin K (K1, K2, K3 and their derivatives). Each possibility represents a separate embodiment of the present invention. In specific embodiments the vitamin is vitamin D.

In other embodiments, the HN is an unsaturated fatty acid, including but not limited to linoleic acid, conjugated linoleic acid (CLA), omega-3 fatty acids such as alpha linolenic acid, docosahexaenoic acid (DHA) and eicosapentaenoic acid (EPA) and their glycerol-esters or other esters. Each possibility represents a separate embodiment of the present invention. In specific embodiments the unsaturated fatty acid is CLA.

In other embodiments the HN is a sterol, cholesterol or its derivatives. In other embodiments, the HN is a carotenoid including α-, β-, or γ-carotene, lycopene, lutein, zeaxanthin, astaxanthin and others. In some embodiments the HN is selected from phytochemicals, phytoestrogens including phytosterols (e.g. β-sitosterol, campesterol, stigmasterol etc.), isoflavones (genistein, daidzein), stilbenes (e.g. resveratrol, trans-resveratrol), lignans (e.g. Matairesinol) and coumestans (e.g. coumestrol), curcumin, and others. In another embodiment the HN is coenzyme-Q10 (co-Q10). Each possibility represents a separate embodiment of the present invention. In some embodiments, the nutraceutical is water soluble. The nutraceutical may be selected from a polyphenol (e.g., punicalagin), a tannin, a catechin, a flavonoid, an isoflavonoid or a neoflavonoid. Non-limiting examples are epigallocatechin gallate (EGCG), epicatechin (EC), epicatechin gallate (ECG), and epigallocatechin (EGC). Each possibility represents a separate embodiment of the present invention. In specific embodiments the nutraceutical is epigallocatechin gallate (EGCG).

In some embodiments, the nutraceutical is an amphiphilic nutraceutical, i.e., it is a chemical compound comprising both hydrophobic and hydrophilic moieties. Any type of protein or peptide, and reducing polysaccharide or oligosaccharide may be used to form the protein-polysaccharide conjugates of the present invention.

The protein in the conjugate may be a vegetable protein, an animal protein, a milk protein, an egg protein, a fungi protein, a microbial protein, an algae protein or any hydrolyzate, peptide or combinations thereof.

In some embodiments, the protein is a vegetable-derived protein, such as but not limited to rice protein, soy protein, pea protein, lupin protein, Zein (corn protein), wheat protein, gluten, and their hydrolyzates. Non-limiting examples of soy proteins are beta-conglycinin and glycinin. In one specific embodiment, the vegetable protein is rich protein hydrolyzate (RPH). In another specific embodiment, the vegetable protein is beta-conglycinin (β-cong).

In other embodiments, the protein is an animal-derived protein. In other embodiments, the protein is a dairy (i.e., milk)-derived protein, such as but not limited to casein, whey protein concentrate (WPC), and whey protein isolate (WPI). In one specific embodiment, the milk protein is casein (which may be in the form of sodium caseinate or an isolated casein such as but not limited to alpha s1, alpha s2, beta or kappa casein, or any combination thereof). In some embodiments the source of casein is sodium caseinate. In other embodiments the source of casein is milk, or milk powder, or any soluble caseinate or casein preparation, or isolated alpha, beta, and/or kappa casein or mixtures of such caseins. In other embodiments, the fungi protein is a hydrophobin. Each possibility represents a separate embodiment of the present invention.

In other embodiments, the protein is any combination of the above vegetable, animal or dairy (milk) proteins, or their hydrolyzates.

The novel nanoencapsulated compositions of the present invention can be introduced into any beverage product to provide a clear solution. Non-limiting examples of beverages include water, soft drinks, juice, milk, tea and coffee.

A certain possible characteristic of the Maillard-based conjugates is their brown color, which may in some embodiments be utilized as a natural pigment for certain beverages.

In other embodiments, the present invention provides methods for the enrichment of beverages with at least one nutraceutical, comprising the step of adding to a beverage a nutraceutical encapsulated by a covalently bonded protein (or peptide)-polysaccharide (or oligosaccharide) conjugate (PPC), wherein the particle size of said composition is sufficiently small such that, when added to said beverage, a clear solution is formed. In one embodiment, the PPC is formed by a Maillard reaction. In another embodiment, the clear solution has an absorbance of less than about 0.1 at 600 nm.

In yet another aspect the present invention provides a method for the preparation of a composition comprising a nutraceutical encapsulated by a covalently bonded protein (or peptide)-polysaccharide (or oligosaccharide) conjugate (PPC) as described herein. The method comprises the following steps:
i) preparing a solution comprising a nutraceutical in water or in a water-miscible solvent, such as ethanol;
ii) preparing solution comprising a covalently-bonded protein (or peptide)-polysaccharide (or oligosaccharide) conjugate (PPC); and
iii) mixing the nutraceutical solution with the PPC solution.

PPCs can be formed as described in, e.g., Nursten et al, the contents of which are incorporated by reference herein.³¹ Preferably, the mixing step (iii) occurs comprises slowly adding the nutraceutical solution to the PPC solution while stirring. In some embodiments the method further comprises the step of drying the encapsulated composition. In other embodiments, the method further comprises the step of homogenizing the dried composition so as to reduce the particle size to the desired range.

The solvent used to prepare the nutraceutical or PPC solution can be any food grade solvent. When the nutraceutical is a HN, a water miscible organic solvent which evaporates during the drying of the conjugates is preferably used. Natural or synthetic solvents known in the art can be used according to the teachings of the present invention. In some embodiments the solvent is ethanol. When the nutraceutical is a hydrophilic compound, water also may be used as a solvent.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description, and are thus included within the scope of this invention.

### BRIEF DESCRIPTION OF THE FIGURES

FIGS. 1A-1D: represent SDS-PAGE patterns of RPH-MD conjugates formed by the Maillard reaction as a function of time and pH. (A) pH 4.5; (B) pH 7.5; (c) pH 8.8 and (d) pH 10.5. Below each lane, the sample composition and the day of heating are specified. S.M.=size marker; the molecular weights (kDa) of the standards are indicated. The loaded protein amount was 1 mg.

FIG. 2: Browning during Maillard reaction in RPH-MD (A), and RPH (B) samples.

FIG. 3: depicts an estimation of residual free amino groups by OPA analysis for RPH-MD samples as a function of heating time at different pH conditions.

FIG. 4: shows the absorbance spectra of RPH (A) and RPH-MD (B) under initial pH 7.5 at 0-4 days of heating (lines from bottom up represent day 0 to 4).

FIG. 5: Absorbance change at 350 nm for the RPH-MD and RPH samples, as a function of the heating time.

FIG. 6: DLS size distribution of RPH and RPH-MD samples at concentration of 2% (on a protein basis). The resulted peaks values are: ˜1 nm for the unheated samples, 1.4 nm for the heated RPH, 2.1 for the Heated RPH-MD.

FIG. 7: (A) Average particle diameters obtained by DLS for CLA in PBS pH 7 (blank) or in 1% protein solutions of the RPH-MD mixture, and conjugate. The CLA, predissolved in ethanol (0.5% final ethanol concentration), was added into the aqueous solutions while stirring. A detailed particle size distribution is displayed at intervals of 0-50, 50-100, 100-200, >200 nm for blank (B), mix (C) and conjugate (D) samples.

FIG. 8: Absorbance of CLA samples in blank (PBS pH 7), or 1% protein solutions: mixed or conjugated RPH and MD. The CLA was added into the aqueous solution to a final ethanol concentration of 2%.

FIG. 9: Average particle diameters obtained by DLS for VD in blank (water), RPH-MD mix or conjugate at 0.1% w/w protein. The ethanol concentration was 4%. Detailed particle size distributions are depicted at intervals of 0-5, 5-50, 50-100, >100 nm for blank (B), mix (C) and conj (D) samples.

FIG. 10: Absorbance (600 nm) of the VD samples in blank (PBS 7), or 1% (w/w) protein media-mix or conjugate. The VD was added into the aqueous solution to a final ethanol concentration of 2%.

FIG. 11: Particle size distribution measured by DLS for conjugate and blank samples with and without homogenization (20-25 kpsi). Sample composition was 0.1% protein, 0.5% ethanol, 0.02 mg/ml VD in a pH 7 PBS.

FIG. 12A: CLA remaining after 1 and 4 days at pH 3.0 and 4° C. CLA concentration was 0.09 mg/ml, and samples contained 0.5% ethanol and 1% protein. FIG. 12B: Protection of VD against acidic pH treatment. Residual VD percent is plotted. The treatment was performed by keeping the samples at pH 2.5 for 2 hr at RT. Initial VD concentration was 0.02 mg/ml, and samples contained also 0.5% ethanol and 0.1% w/w protein.

FIG. 13A: Simulated shelf life study of CLA at RT, in pH 7 PBS, comparing conjugate, mix and blank. CLA concentration 0.09 mg/ml. samples contained 0.5% ethanol and 1% protein. Curve-fits are drawn as first-order approximation. FIG. 13B: The shelf life of VD at RT and 4° C. The solvent PBS pH 7, vitamin D concentration 0.02 mg/ml, 0.5% ethanol, 0.1% protein

FIG. 14A: Particle size distribution of Maillard reaction products in PBS (pH 6.87, 30 mM) at beta-conglycinin (β-cong): MD molar ratios of 1:1, 1:2, 1:4, 1:8 and control sample of heated β-cong. FIG. 14B: Conjugate (β-cong: MD molar ratio 1:8), β-cong-MD mix (β-cong: MD molar ratio 1:8) and β-cong solubility in PBS (pH 6.87, 30 mM).

FIG. 15: Particle size distribution at time 0 (FIG. 15A) and 48 hr (FIG. 15B) of a β-cong-MD conjugate with and without EGCG, mixture solution of β-cong and MD, MD and β-cong with and without EGCG. EGCG concentration: 0.0125% w/v, conjugate/mixture/β-cong concentration: 0.092% w/v, pH=6.69

FIG. 16: Absorbance of EGCG solutions at 425 nm (indication of EGCG oxidation) with the conjugate or with mixture solution of β-cong and MD. EGCG concentration: 0.0125% w/v, conjugate/mixture concentration: 0.092% w/v, pH=6.69.

FIG. 17: solubility of MD DE=6: A1, B1=casein. A2, B2=MD. A3, B3=casein with MD mixture. A4, B4=CN-MD conjugates (heating time 4 hrs)

FIG. 18: SDS PAGE. Lanes: 1-4 conjugates: 1—MD: CN=8, 2—MD: CN=4, 3—MD: CN=2, 4—MD: CN=1; lanes 5-6—mixture: 5—MD: CN=8, 6—MD: CN=1, 7—CN, 8-size marker. Conjugation time=8 hrs.

FIG. 19: Residual amines as a function of MD:Casein molar ratio at different heating times.

FIG. 20: % protein at supernatant (an indication of the yield of separation) at pH=4.6 (pI of casein) as a function of MD:Casein molar ratio.

FIG. 21: Particle size distribution in the supernatant after separation at the pI, and addition of 1 mg/ml VD (CN: VD molar ratio=1:1).

FIG. 22: Size distribution of CN:MD conjugate (FIG. 22A) or mixture (FIG. 22B), each +VD3 at different VD concentrations. Casein concentration was 1 mg/ml and the pH was 7.

FIG. 23: The percentage of residual vitamin D3 after 2 hrs at pH=2.5. Values were normalized according to extraction yields.

FIG. 24: Residual VD2 as a function of time, encapsulated in conjugate, mixture of MD and casein, buffer. All solutions were at pH=7. Casein concentration was 3 mg/ml (0.13 mM). VD concentration was 0.05 mg/ml (0.13 mM). MD: CN molar ratio was 4, VD:CN molar ratio was 1.

FIG. 25: EGCG degradation as a function of time (degradation products absorb at 425 nm), with and without the different protective systems studied. EGCG concentration was 0.9 mg/ml, casein concentration was 5 mg/ml.

FIG. 26: (A) Nile Red (NR) absorbance spectra in different systems. NR concentration=1.4M, Casein concentration was 5 mg/ml. (B) NR emission spectra in different solutions. Ex: 570 nm, slit widths 5, 5. CN conc.=5 mg/ml. NR conc.=1.3 μM.

FIG. 27: detected NR in water and on glass 2 hrs after addition of NR (1.3 μM) to water.

FIG. 28: (A) NR (1.3 μM) with con/mix/buffer with and without pepsin after 2 hrs at 37° C. pH=2.5. (B) % of NR adsorbed to glass in con/mix/buffer solutions, with and without pepsin after 2 hrs at 37° C., pH=2.5.

### DETAILED DESCRIPTION OF THE INVENTION

The present invention provides covalently bonded protein-polysaccharide conjugates (PPC) (including conjugates comprising peptides and oligosaccharides) as vehicles for nanoencapsulation of biologically active compounds, particularly nutraceuticals. The PPCs efficiently encapsulate both hydrophobic (i.e., water insoluble or poorly water-soluble) and hydrophilic (i.e., water-soluble) nutraceuticals, to provide a composition which, when added to a beverage, disperses so as to provide a clear solution. Preferably, the clear solution has an absorbance at 600 nm of less than about 0.1. Advantageously, the conjugates of the present invention enhance the dispersibility of hydrophobic nutraceuticals and protect nutraceuticals from degradation, over a wide range of pH values, both during shelf life and upon gastric digestion. In one embodiment, the PPCs are formed by a Maillard reaction.

The present invention now discloses that co-assembly of biologically active compounds, for example nutraceuticals (which are preferably hydrophobic but can also be hydrophilic or amphiphilic), with PPCs, stabilizes the nutraceuticals and protect them from degradation, even in acidic media such as that found during gastric digestion. These PPC may also protect hydrophilic nutraceuticals against degradation, mainly by oxidation. Such PPC-nutraceutical system facilitates the enrichment of beverage products, while minimizing the effect of the compound incorporation on the beverage properties, and still maintaining its transparency. In one embodiment, the PPCs are Maillard-reaction formed.

Encapsulation of biologically active compounds within PPCs is advantageous over hitherto known encapsulation methods as the compositions comprise only natural components, and their particle size is sufficiently small so as not to form turbid solutions or precipitates when mixed with the beverage of choice. In addition, when the active compound possesses undesirable attributes, the encapsulation in the PPCs diminishes such unwanted features (e.g. in the case of omega 3 fatty acids). Another important potential benefit is the improved bioavailability of the enclosed compound due to its distribution, at a molecular level, over a very large surface area of the PPC-based nanocapsules, and in the case of casein-based PPC, the fact that caseins are evolutionally optimized for ease of digestion and absorption.

Specific embodiments include a method for incorporation of hydrophobic nutraceuticals (HN) such as vitamin D and Conjugated Linoleic Acid (CLA) into rice protein hydrolyzate (RPH)-maltodextrin (RPH-MD) conjugates. Other embodiments include a method for protection of the water-soluble nutraceutical epigallocatechin gallate (EGCG), as well as the hydrophobic nutraceutical vitamin D (VD) using Soy beta-conglycinin-MD Maillard conjugates. Other embodiments include a method for the incorporation of hydrophobic nutraceuticals (HN) such as vitamin D and casein-maltodextrin (CN-MD) conjugates. The methods of the present invention further included the evaluation of the encapsulation processes as well as the protection conferred to the nutraceuticals by the encapsulation process and or by the conjugates themselves.

For example and as disclosed herein for the first time, vegetable proteins (e.g., Rice, Soy or their hydrolyzates), or milk proteins (e.g., casein (CN)), were conjugated to an oligosaccharide (e.g., Maltodextrin (MD)) or polysaccharide using the Maillard reaction or Maillard-type reaction by dry heating (60° C., 79.9% RH for several hours to days). The formation of conjugates was verified by SDS-PAGE, decrease of free amino groups by the o-Phthalaldehyde (OPA) assay, visual color test, DLS, and spectrophotometric absorbance showing increase of peaks at the wavelength region typical for the Maillard products. The conjugation products showed an increase in molecular weights as a function of time with similar reaction rate for pH 4.5, 7.5, 8.8 and significantly higher initial rate for pH 10.5, where most conjugation occurred on the 1^{st} day of heating.

In one embodiment, the co-assembly of rice protein hydrolyzate (RPH)-maltodextrin (RPH-MD) conjugates with vitamin D (VD) or with conjugated linoleic acid (CLA) was examined by mean of DLS particle size distribution and turbidity measurements, and a significant diameter decrease and turbidity reduction were observed in the presence of RPH-MD, indicating the interaction and solubilization effect exerted by RPH-MD conjugates. The degradation of CLA and VD during shelf life at both 4° C. and room temperature, at both neutral and acidic conditions was significantly slower due to nanoencapsulation with RPH-MD, suggesting Maillard conjugates of RPH-MD can serve as nano-vehicles for delivery of HN such as CLA or VD in transparent aqueous systems providing protection against degradation.

In other embodiments, soy beta-conglycinin-MD Maillard conjugates showed better solubility than the mixture of their components. The conjugates, with and without the nutraceutical EGCG gave smaller particle sizes than solutions of MD with and without EGCG, forming clear solutions. The protection provided by the conjugate-based nanoparticles to EGCG was more significant than the protection provided by the simple beta-conglycinin-MD mixture or control sample. These results emphasize the potential of soy beta-conglycinin-MD Maillard conjugates as protective material for clear drink applications.

In another example, for enrichment of clear beverages with a hydrophobic nutraceutical (e.g. vitamin D), CN-MD Maillard conjugate based nanovehicles having diameters of less than 100 nm, were formed. At high VD concentrations (simulating soft drink concentrates), the complexes of VD-conjugate were less turbid than the ones formed by VD and a CN-MD mixture (where each biopolymer was heated separately, then mixed) and much less turbid than VD dispersed in buffer only. Completely clear solutions were obtained with nanoencapsulated VD at doses typical for the final drinks). An industrially feasible fractionation process was developed based on isoelectric precipitation, for enrichment of clear beverages even at pH close to 4.6, the pI of the native casein, where casein nanocapsules would precipitate. Conjugation significantly improved the protection against oxidation conferred to both VD and EGCG. Nanoencapsulation of VD in CN-MD Maillard conjugates conferred significant protection against low pH induced degradation, important for acid drinks, and for survival through gastric digestion. This attribute may be utilized for developing targeted vehicles for enteric delivery of bioactives and drugs.

Overall the study showed the very good potential of Maillard conjugates of proteins and oligosaccharides for nanoencapsulation of nutraceuticals for clear drink applications at high encapsulation efficiency (possibly as high as ˜90%), good solubilization, stabilization and protection conferred to the sensitive bioactive compound against degradation during shelf life, and gastric digestion.

The concentration of the nutraceutical in the PPC can vary depending on the nature of the nutraceutical and its function. Typical concentrations can vary between 0.01 to 100 mg/ml, for example 0.01 to 10 mg/ml, 0.01 to 5 mg/ml or 0.01 to 1 mg/ml.

All references cited herein are hereby incorporated by references in their entirety as if fully set forth herein.

### DEFINITIONS

For convenience and clarity certain terms employed in the specification, examples and claims are described herein.

The terms “transparent” as used herein means having the property of transmitting rays of light through its substance so that bodies situated beyond or behind can be distinctly seen. A “clear solution” as used herein means a transparent solution. The term “turbidity” or “turbid” as used herein is the cloudiness or haziness of a fluid caused by scattering of visible light by particles (suspended solids or liquids) that are individually generally invisible to the naked eye. Turbidity can be measured by measuring absorbance at an appropriate wavelength (usually 600 nm is used). Absorbance at 600 nm below 0.1 is generally typical of transparent systems, below 0.05 is typically considered good transparency, and below 0.02 is typically considered excellent transparency. Thus, the solutions of the present invention typically will have an absorbance at 600 nm of below about 0.1, preferably below about 0.075, preferably below about 0.05, more preferably below about 0.02 and even more preferably below about 0.01.

The terms “poorly water-soluble” or “hydrophobic” refer to water solubility of less than about 30 mg/ml, less than about 10 mg/mL, or less than about 1 mg/mL at ambient temperature and pressure and at about pH 7. This corresponds to nutraceuticals which are to be characterized by the commonly used terms “sparingly soluble”. “slightly soluble”, “very slightly soluble”, “practically insoluble” and “insoluble”, all of which are used herein interchangeably.

### Nutraceuticals:

A “nutraceutical”, also known as a functional food (or its component), is generally any one of a class of food ingredients or dietary supplements including vitamins, minerals, herbs, healing or disease-preventative foods or food components that have medical or pharmaceutical effects on the body. Examples of non-polar or hydrophobic nutraceuticals include, but are not limited to fatty acids (e.g., omega-3 fatty acids, DHA and EPA or their esters); fruit and vegetable extracts; vitamins A, D, E and K; phospholipids, e.g. phosphatidyl-serine; certain proteoglycans such as chondroitin; certain amino acids (e.g., iso-leucine, leucine, methionine, phenylalanine, tryptophan, and valine); various food additives, various phytonutrients, for example lycopene, lutein and zeaxanthin; certain antioxidants; plant oils; fish and marine animal oils and algae oils. It is to be understood that certain nutraceuticals can also be referred to as therapeutics as well as cosmetic compounds.

Some non-limiting examples of hydrophobic nutraceuticals include, but are not limited to:

(a) Fat-soluble vitamins including vitamin D (D2, D3 and their derivatives), vitamin E (α, β, γ, δ-tocopherols, or α, β, γ, δ-tocotrienols), vitamin A (retinol, retinal, retinoic acid), vitamin K (K1, K2, K3 and their derivatives).

(b) Unsaturated fatty acid, including but not limited to linoleic acid, conjugated linoleic acid (CLA), omega-3 fatty acids including alpha linolenic acid, docosahexaenoic acid (DHA) and eicosapentaenoic acid (EPA) and their esters, including their glycerol esters.

(c) A sterol, cholesterol or its derivatives.

(d) Carotenoids including α-, β-, or γ-carotene, lycopene, lutein, zeaxanthin, astaxanthin and others.

(e) Phytosterols (e.g. β-sitosterol, campesterol, stigmasterol etc.), isoflavones (e.g., genistein, daidzein), stilbenes (e.g. trans-resveratrol), lignans (e.g. Matairesinol) and coumestans (e.g. coumestrol), and others.

(f) Polyphenols (e.g., punicalagin), tannins.

In yet other embodiments the nutraceutical is selected from a bioactive peptide, such as casein-phosphopeptide (CPP) and other calcium-binding peptides.

Nutraceuticals for use in the compositions of the present invention may also be hydrophilic (i.e., water soluble). In such embodiments, the nutraceutical is selected from a catechin a flavonoid, an isoflavonoid or a neoflavonoid. Non-limiting examples are epigallocatechin gallate (EGCG), epicatechin (EC), epicatechin gallate (ECG), and epigallocatechin (EGC).

(i) Vitamin D (VD):

VD is an oil-soluble vitamin which is photochemically synthesized in the skin during exposure to ultraviolet radiation (UVB) of mid-day sunlight. It is crucial for multi-system function: calcium and bone metabolism, muscle function, insulin reactivity, cell differentiation, immune system function and more. Adequate VD status was linked to reduced risks for fractures, hypertension, diabetes, cancer and more. VD status is far below optimal in many countries all over the world, mainly due to avoidance of sun exposure to prevent melanoma, and the use of sunscreen which blocks VD synthesis. Also, the nutritional sources of VD are scarce, and cannot provide sufficient amounts when sun exposure is lacking. Therefore, it is imperative to enrich staple foods and drinks with VD to raise its consumption by large populations.

VD tends to oxidize readily in aqueous solution and especially under acidic conditions. The structures of the two most prominent forms of vitamin D, vitamin D2 and vitamin D3, are shown below:

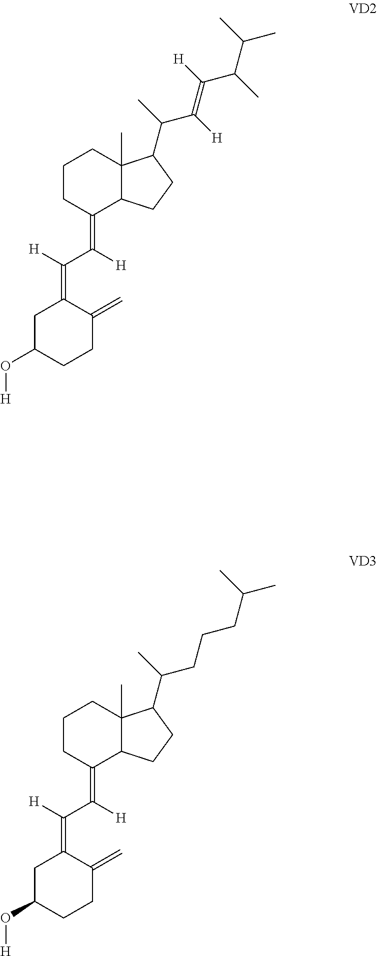 [SMILES: C=C1CCC(O)C/C1=C/C=C1\CCCC2(C)C1CCC2C(C)/C=C/C(C)C(C)C ; C=C1CC[C@@H](O)C/C1=C/C=C1\CCCC2(C)C1CCC2C(C)CCCC(C)C]

(ii) Conjugated Linoleic Acid (CLA)

CLA is mainly found in meat and milk products from ruminant animals. CLA has been attributed with diverse health benefits which include immune response enhancement, atherosclerosis reduction, growth enhancement, anti-diabetic, anti-atherogenic and antiadipogenic properties. In addition, it has been reported that CLA can inhibit the proliferation of various cancer cell lines and act as an inhibitor of chemically induced carcinogenesis.

Both VD and CLA are hydrophobic compounds that readily dissolve in oil or organic solvents but have very poor water solubility. When added to water, VD and CLA provide unstable turbid suspensions. Moreover, both of the compounds are subjected to oxidative processes, which lead to loss of its bioactivity and to decreased nutritional quality when it is used as a food additive. Vitamin D was found to be very unstable in aqueous solutions and even more so in acidic conditions. Because of the presence of conjugated double bonds in the molecular structure of CLA, its oxidative stability was shown to be extremely low. As demonstrated herein, the Maillard protein-polysaccharide conjugates used as nanovehicle formers according to the principles of the present invention helped solubilize these and other HN in stable transparent solutions, while protecting them from various degradation reactions.

(iii) Epigallocatechin-3-Gallate EGCG:

EGCG is the major catechin found in green tea, comprising 50%-60% of the total catechin mass. EGCG is a water soluble compound, readily oxidized at neutral and alkaline pH, and degraded to yellow products absorbing visible light at wavelength of 425 nm. Animal studies indicated that the consumption of green tea and green tea products with high levels of EGCG and other catechins may have a significant effect toward the prevention of tumors, cardiovascular disease, neurodegenerative disease, obesity and other adverse medical conditions. The chemical structure of EGCG is shown below:

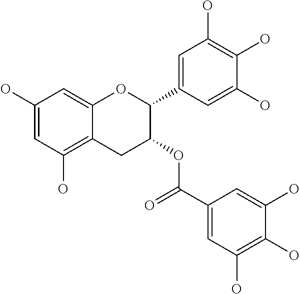 [SMILES: O=C(O[C@@H]1Cc2c(O)cc(O)cc2O[C@@H]1c1cc(O)c(O)c(O)c1)c1cc(O)c(O)c(O)c1]

### Caseins (CN):

As used herein, the term “casein” refers to the predominant protein in milk, comprising the subgroups αₛ₁, αₛ₂, β and κ.

Casein is organized in micelles. Casein micelles (CM) are designed by nature to efficiently concentrate, stabilize and transport essential nutrients, mainly calcium and protein, for the neonate (1). All mammals' milk contains casein micelles. Cows' milk contains 30-35 g of protein per liter, of which about 80% is casein.

Casein micelles are spherical colloids, 50-500 nm in diameter (average of 150 nm) (2), made of the main four caseins: αₛ₁-casein (αₛ₁-CN), αₛ₂-CN, β-CN, and κ-CN (molar ratio ˜4:1:4:1 respectively). The caseins are held together in the micelle by hydrophobic interactions and by bridging of calcium-phosphate nanoclusters bound to serine-phosphate residues of the casein molecules. The structure of the casein micelles is important for their biological activity in the mammary gland as well as for their stability during processing of milk into various products, as well as for the good digestibility of the nutrients comprising the micelles. The micelles are very stable to processing, retaining their basic structural characteristics through most of these processes.

The choice of casein for use as the protein part for the conjugates of the present invention stems from the excellent amphiphilicity of caseins, their low price, and their large number of side-amine residues. An average casein molecule contains 13.6 lysine (Lys) residues, which may theoretically serve as Maillard-conjugation sites.

### Maltodextrin

Maltodextrin (MD) is an oligosaccharide formed by hydrolysis of starch. Hydrolyzates of starch are characterized by the “dextrose equivalent” (DE) value. DE is defined as the percentage of the total solids that have been converted to reducing sugars following starch hydrolysis. MD is defined as hydrolyzed starch having DE of 3-20. The higher the DE is, the lower the average molecular weight is and thus the MD is more easily dissolved in water. Starch consists of D-glucose monomers linked with an α(1-4) glycosidic linkage. There are two types of starch: amylose—a linear form consisting mainly of α(1-4) linked glucose units, and amylopectin—a branched form of starch, wherein the side chains are linked to the backbone via an α(1-6) linkage.

Due to the branching, the density of amylopectin is lower than the density of amylose. MD derived from corn starch was used in some embodiments of the present invention since corn starch contains about 70% amylopectin—a branched form of starch, to obtain a larger hydrophilic part of the conjugate. This is advantageous for steric repulsion, and for increasing the curvature of the nanoparticles formed—so that their radii would be smaller. A MD molecule has only one reducing end, which is a desired feature, so that the conjugation would not lead to gelation, but only to low molecular weight copolymers.

### The Maillard Reaction

A scheme of the initial stages of the Maillard reaction is shown in Scheme 1:³¹

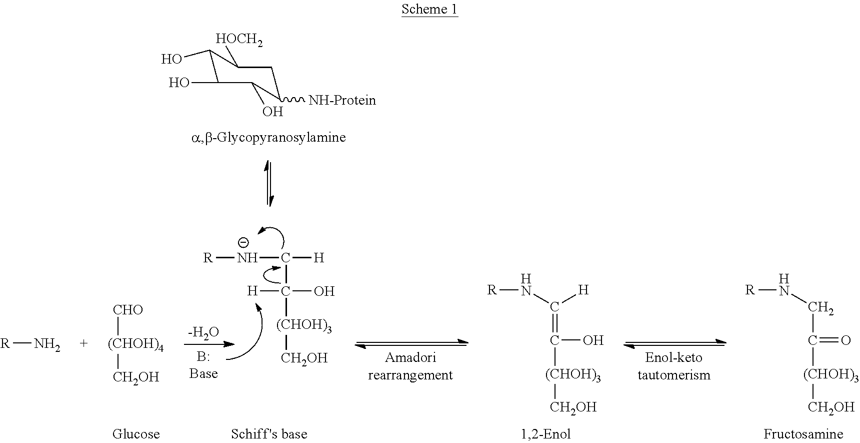

See, Reference 31 for a description of the Maillard reaction. The initial stage includes two reactions: 1. Amine-sugar condensation in which a covalent bond is formed between an amine of a protein and a reducing end of a saccharide, a water molecule is released, and a Schiff base is formed. 2. Amadory rearrangement in which a series of isomerizations form an Amadory compound.

During the initial stage, the products remain as whole saccharide-protein copolymers. In most cases lysine is the most reactive residue in proteins, while Tryptophan and Arginine are less reactive. The condensation reaction is affected by the water activity (a_{w}). The reaction rate is accelerated at a_{w} between 0.5 and 0.8, while at lower a_{w} values the reactants lose their mobility, and at higher a_{w} they are diluted. [19] The reaction rate also increases with increasing pH for two reasons: the reactive form of the amine group is the unprotonated form, and the reactive form of the sugar is the open chain form, which is more prevalent at higher pH conditions. [38] The formation of Schiff base was found to be rate limiting. The formation of Amadory compounds was found to be the most temperature sensitive reaction of the initial stage. The Amadory reaction rate rises sharply at temperatures above 70° C. and at pH above 8. [19] To gain mostly products of the initial stage with minimum degradation and\or polymerization (typical of later stages), reaction conditions should be kept below 70° C. and pH<8.

A scheme of the intermediate and progressive stages of the Maillard reaction is shown in Scheme 2:³¹

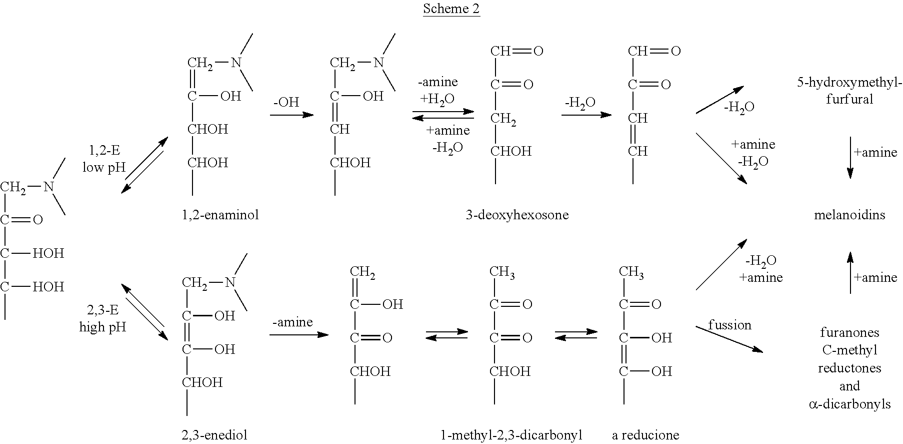

The intermediate stage includes three reactions: 1. An exit of a water molecule from the saccharide. 2. Breakdown of the saccharide. 3. Strecker degradation—a degradation of the amino acid. During this stage the saccharide-protein copolymer breaks, free radicals form and react. Because free radical formation is involved the reactions are highly non-specific.

In the finals stage fragments are polymerized to form melanoidins—a high molecular weight polymer compounds. The routes of degradation and polymerization are greatly affected by the pH.

It has been reported that during the Maillard reaction, antioxidants are formed, which is important for the functionality of Maillard reaction conjugates as protective nanoencapsulators for oxidation sensitive nutraceuticals.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### List of Abbreviations

a_{w}—water activity

BSA—Bovine serum albumin

CN—Casein

Conjugated linoleic acid (CLA)

DE—dextrose equivalent

DHA—Docosahexaenoic acid

DLS—Dynamic light scattering

EGCG—Epigallocatechin-3-gallate

Em—Emission

EPA—Eicosapentaenoic acid

Ex—Excitation

GRAS—Generally recognized as safe

HN—Hydrophobic nutraceutical

Lys—Lysine

M—Molar

MD—Maltodextrin

MW—molecular weight

NR—Nile red

O.D.—Optical density

OPA—Ortho-phthaldialdehyde

PAGE—Polyacrylamide gel electrophoresis

PPC—Protein polysaccharide conjugate

RH—Relative humidity

RP-HPLC—reversed phase high performance liquid chromatography

SDS—Sodium dodecyl sulfate

UV—Ultraviolet

VD—Vitamin D

VIS—Visible

### I. Nanocapsules Made of Maillard Reaction Based Conjugates of Vegetable Proteins and Maltodextrin

### Example 1

### Rice Protein Hydrolyzate and Maltodextrin Based Nano Vehicles for Nutraceutical Delivery

The objective of this experiment was to form Maillard conjugates of Rice Protein Hydrolyzates (RPH) and maltodextrin (MD), characterize them and evaluate their potential for nanoencapsulation of hydrophobic nutraceuticals, preferably for clear liquid systems.

Materials

Maltodextrin (MD) of dextrose equivalent (DE) 19 with an average molecular weight of approximately 10 KDa, was donated by Productos de maiz S.A. Corn Products international (Munro, Argentina). RPH was donated by Cognis Ltd. (Dusseldorf, Germany). It was produced by specific proteolysis resulting in peptides of 2-3 KDa, and obtained in a form of a 30% solution, preserved with a sodium benzoate, at pH 4.5.

O-phthaldialdehyde (OPA), Trizma base, SDS, vitamin D3 (VD), conjugated linoleic acid (CLA), mercaptoethanol, acrylamide-bisacrylamide mixture, and ammonium persulphate were obtained from Sigma-Aldrich (Rehovot, Israel). Methanol and Acetonitrile, both of HPLC grade, were obtained from LabScan (Dublin, Ireland). NaOH was obtained from Merck (Darmstadt, Germany), absolute ethanol from BioLab (Jerusalem, Israel), and sodium tetraborate from Laba Chemie (Mumbai, India). SDS-PAGE size markers and coomassie brilliant blue 250-R stain were obtained from Bio-Rad. Bromophenol blue was obtained from Fluka.

The pH 7 PBS buffer comprised 30 mM NaH₂PO₄/Na₂HPO₄. The pH 3 citrate buffer comprised 30 mM sodium citrate. 0.02% (w/w) sodium azide was added to the buffers as a preservative.

Methods

### Conjugation Process by the Maillard Reaction

A 50 mg/ml solution of RPH and MD at 1:1 w/w ratio was prepared in doubly distilled water (ddw). The pH of the obtained solution was 4.5; pH values of 7.5, 8.8 and 10.5 were obtained by adding 1M NaOH. The control solutions of the RPH without MD were prepared at the same conditions. The solutions were then freeze-dried, and placed in an oven at 60° C. A constant relative humidity of 79.9% was achieved by placing the samples in a desiccator containing saturated KBr solution. Samples were removed from the oven every 24 hr during 0-5 days, freeze-dried again and stored for further analysis.

Entrapment of Hydrophobic Nutraceuticals (HN) within the RPH-MD Micellar Nanoparticles.

The co-assembly of the HN with the RPH-MD was achieved by slowly adding the VD or CLA, dissolved in ethanol, into the RPH-MD solution during stirring. The HN ethanol solutions were prepared at different concentrations while the final ethanol concentration was kept constant and did not exceed 4%.

### SDS-PAGE Analysis

The Maillard reaction products were analyzed by electrophoresis method based on Tricine-SDS-PAGE, for low Mw peptides resolution. Three-layer gel was prepared as follows: the lower separating gel was composed of 16.5% of acryl amide:bisacrylamide (19:1), the intermediate gel—10% acryl amide:bisacrylamide (29:1) and the upper stacking gel—4% acryl amide:bisacrylamide (29:1). The sample buffer contained 24% (v/v) glycerol, 1% SDS, 0.6 Tris and ˜2 mg/ml bromophenol blue. The samples in a form of lyophilized powder were dissolved in the sample buffer. Then mercaptoethanol was added to a final concentration of 5% (v/v) and the samples were incubated for 5 min at 100° C. while vigorously stirring. Final sample concentration of 50 mg/ml on a protein basis was obtained. A volume of 20 μl was loaded into the gel wells. The voltage was adjusted to 30 V during the first hour of electrophoresis and to 150 V during the next two hours. After the electrophoresis the gels were immersed in a fixation solution (30% methanol, 10% acetic acid) for 0.5 hr, then stained in a coomassie brilliant blue R-250 for 1 hr and washed by 10% acetic acid.

### Estimation of Conjugation Degree by OPA Assay

The RPH-MD graft samples were analyzed by the OPA assay to determine the degree of conjugation. The OPA reagent was prepared as described in the literature.^{33,34} The following compounds were diluted with water to 100 ml: 80 mg OPA (dissolved in 2 ml 95% ethanol); 50 ml 0.1 M sodium tetraborate, 5 ml 20% SDS; 0.2 ml of 2-mercaptoethanol. The OPA reagent was prepared immediately before use. The RPH-MD samples at a concentration of about 0.1 mg/ml on protein base (taking the RPH Mw as 2.5 KDa, this concentration corresponds to 4E-5 M) were prepared in DDW with 0.02% azide. 0.05 ml of the sample was added to 2 ml of OPA reagent. This solution was briefly stirred and the absorption at 340 nm was measured after 2-min incubation at room temperature (RT). A standard curve was obtained by using L-leucine as a reference compound. Reference samples with a concentration ranging from 1.5E-4 to 1.5E-3 M were prepared in DDW with 0.02% sodium azide and the L-leucine determination was performed as described above.

### Particle Size Distribution Analysis by DLS

The particles size distribution analysis was performed by dynamic light scattering (DLS) analyzer (NICOMP_{—}380, Particle Sizing Systems Inc., Santa Barbara, Calif., USA) as described in previous publications.³⁵

The conjugate samples were characterized as follows. Two mg/ml (on a protein base) solutions were prepared from the dry heated samples of RPH with and without MD and compared to the unheated control. The RPH-MD powder was dissolved in a pH 7 PBS and stirred overnight for complete hydration. The samples were filtered through 0.45 μm syringe filters (polyvinylidene fluoride, Durapore filter (Millipore, Carrigtwohill, Co. Cork, Ireland).

Additionally, the HN RPH-MD co-assembled particles were analyzed at different concentrations of the VD or CLA.

### Turbidity Measurements

The HN samples turbidity was estimated by absorbance measurements at 600 nm using an Ultrospec 3000 spectrophotometer (GE Healthcare, Waukesha, Wis., USA) or the Synergy HT Multi-Detection Microplate Reader.

### VD and CLA Protection During the Shelf Life

The protection conferred by the RPH-MD against HN deterioration with time was evaluated. Different shelf life conditions of temperature 4° C. or 23° C. and pH, neutral or acidic, were tested. Neutral pH conditions were obtained by a pH7 PBS. Acidic conditions for CLA were obtained by a pH 3 citric buffer. Acidic conditions (pH 2.5) for VD were obtained by addition of HCl.

Following the incubation of HN co-assembled samples under the above shelf life conditions, HN extraction was carried out. Then the HN concentration in the samples was quantified using reversed phase HPLC (RP-HPLC), 4.6×100 mm C18-C2 column, on an Akta basic HPLC system equipped with a 3 simultaneous wavelengths UV detector (GE Healthcare, Waukesha, Wis., USA). The volume of the injection loop was 100 and the operation temperature was 24° C.

The concentration of the vitamin D was 0.02 mg/ml, a high dosage intended for simulating beverage concentrates, which are later diluted during bottling, but must be colloidally and chemically stable. The CLA concentration was 0.1 mg/ml which is the highest concentration that could be achieved in visually transparent samples with the help of the conjugates. A molar ratio of HN to RPH of about 1:1 was chosen.

### VD Analysis

Vitamin D extraction and HPLC quantification was based on Kazmi et al⁵⁷. Samples (1 ml) were placed into 12 ml test tubes, followed by the addition of 3.75 ml of a methanol:chloroform mixture (2:1). The tubes were vortexed and 1.25 mL of chloroform were added to each tube, which was again vortexed for 1 min. Samples were centrifuged for 10 min at 1500×g and 4° C. The clear chloroform layer at the bottom of each test tube was collected using a glass syringe and transferred to an evaporation vial. The chloroform extract was dried under a flow of nitrogen gas, reconstituted in 2 mL of the mobile phase [see composition below], and left undisturbed for at least 15 min. Operating conditions were: ambient temperature (24±1° C.); mobile phase was methanol: acetonitrile: water (49.5:49.5:1 v/v); flow rate was 0.3 ml/min; and the absorbance was measured at 254 nm and 228 nm.

### CLA Determination

To extract CLA, isopropanol (0.5 ml) was added to 0.5 ml sample. After vortexing for 30 s, hexane (1 ml) was added, and the tube was vortexed again for 15 min. Then, the samples were centrifuged (1,900×g for 5 min), and the upper hexane layer was collected. Then the hexane was evaporated by nitrogen (99.997% purity) and the concentrate was re-dissolved in 2 ml of acetonitrile/0.14% of acetic acid (vol/vol). Aliquots of the latter were injected into the HPLC system. The separation of CLA was performed with a mobile phase of acetonitrile/water/acetic acid (70/30/0.12, v/v/v) at a flow rate of 1.5 ml/min and CLA was detected at 234 nm, which was found optimal in a spectrum scan (not shown).

### Studying a Homogenization Process for Further Particle Size Reduction

The dispersion of VD with and without RPH-MD was homogenized by Micro DeBee (Bee International Inc. South Easton Mass., USA) ultra-high pressure homogenizer: process pressure: 20-25 kpsi, Orifice diameter: 0.1 mm. The sample composition was 0.1% protein, 0.5% ethanol, VD concentration 0.02 mg/ml, buffer PBS pH 7. The sample volume subjected to homogenization was 25 ml. The influence of the homogenization process on particles size was evaluated by DLS measurements performed 2 hr after the homogenization.

### Results and Discussion

### Studying the Effect of pH on RPH-MD Conjugation by the Maillard Reaction

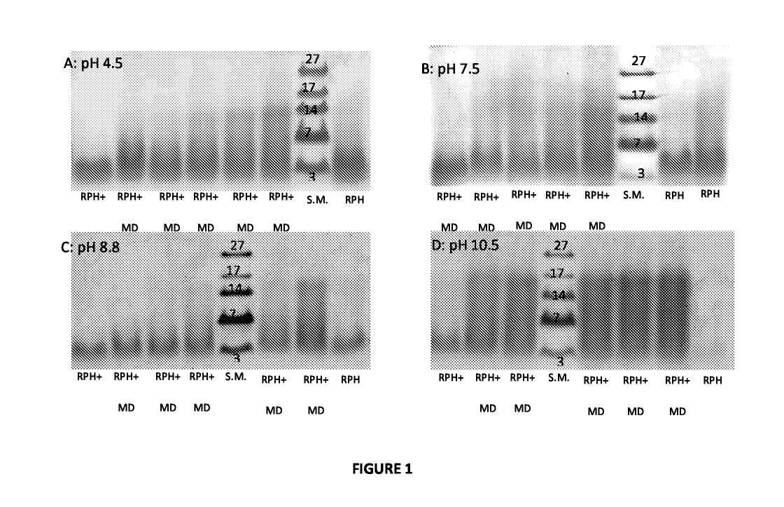

SDS-PAGE analysis was used to verify the conjugation between the RPH and MD, and study the effect of pH on this process. This is a method of choice, as it separates by molecular size, thus enables to visualize higher molecular weight block-copolymer conjugates formed during the Maillard reaction of the saccharides and protein molecules. FIG. 1 displays the results of conjugation as a function of heating time and pH. (B) The Maillard reaction progress is visualized at pH 7.5 during 0-4 days of heating. It can be clearly observed that the RPH-MD samples give higher molecular weight bands with time of heat treatment. A certain smear of the band was observed for the control sample of RPH without MD after 4 days of heat treatment compared to the unheated (time 0) control RPH. This may be explained by the Maillard reaction of saccharide impurities found at about 5% (w/w) in the raw RPH.

Additional pH conditions of 4.5, 8.8 and 10.5 are shown in FIGS. 1A, C and D respectively. In all cases a molecular weight increase with time of heating is evidenced. The time-zero band falls between 2-5 kDa whereas it was expected to be 2-3 kDa according to the manufacturer specification. For pH 4.5, 7.5 and 8.8 the bands front reaches the region of 14-17 kDa. The smeared bands indicate the size distribution of the conjugates, which is expected for such a diverse mixture of peptides, and MD oligomers. The intensity of the color along the band is indicative of the molecular weight increase with time. In all pH conditions, within 4 days of heating the Maillard reaction progressed to a significant extent. However, while pHs 4.5, 7.5 and 8.8 resulted in very similar SDS-PAGE time dependent patterns, pH 10.5 showed notably different behavior. At pH 10.5, the upper front of the band reaches a higher molecular weight than at other pH conditions. Furthermore, contrary to gradual Mw increase at pH 4.5, 7.5 and 8.8, a steep Mw enlargement can be seen for pH 10.5 already after 1 day of heating. Without wishing to be bound by any particular mechanism or theory, it is contemplated that the high alkaline conditions at this high pH speed up the kinetics of the Maillard reaction, as the amine is not ionized. Consistent with this hypothesis, it has been previously disclosed that the rate of Amadori compound formation (the intermediate product leading to quick melanoidin formation, responsible for the brown color) is roughly leveled between pH 2 and 8, but increases at pH 10, and very significantly at pH 12. Another finding is that the band of RPH with no MD almost completely disappeared after 5 days of heat treatment at pH 10.5. It has been reported that heating at extreme alkali conditions causes protein degradation which can explain the weakening of the relevant band. Interestingly, at pH 10.5 after 5 days of the heat treatment the proteins were not harmed in the presence of the MD, suggesting a protective effect of the saccharides on the proteins.

The gel image was analyzed by the ImageJ, Java image processing software and the protein-to-conjugate conversion yield was evaluated according to the following procedure. The initial band area is spread between 2 and 5 kDa. On the final day of the heat treatment the band staining was distributed quite evenly between 2 and 15.7, 16.3, 19, 24 kDa for pH 4.5, 7.5, 8.8, 10.5, respectively. The conjugation yield was evaluated from the band area growth. The resulting yields were 65, 66, 71 and 77% for pH 4.5, 7.5, 8.8 and 10.5, respectively.

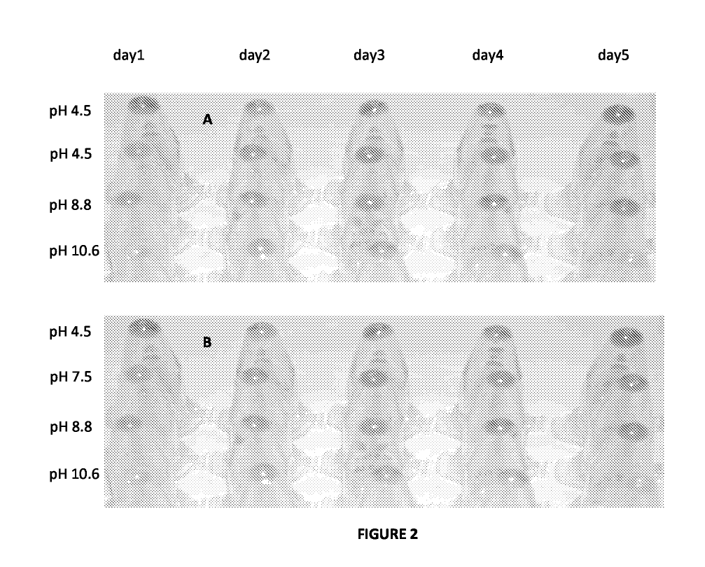

The reaction of RPH with MD resulted in browning which intensified with heating time (FIG. 2A). The color developed more rapidly at pH 10.5, which is an accord with SDS-PAGE results. The control samples of RPH alone were treated at the same condition and photographed for color development comparisons (FIG. 2B). A slightly yellow color was observed. This is typical for the RPH product and the color does not change significantly during the heating time, and as explained above, may be due to interaction with some residual sugars in the raw RPH. The strong darkening observed for samples in the presence of MD is a result of advanced Maillard products (melanoidins) formation.

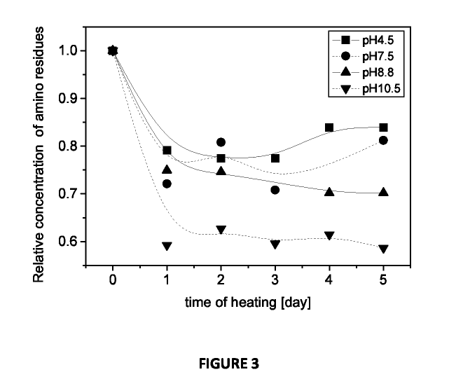

The degree of RPH-MD conjugation was evaluated by quantifying the reduction of free amino groups as was quantified by the OPA assay. The results were summarized in FIG. 3. It can be noticed that the major drop in amino group's concentration occurs during the 1^{st} day of heating after which only a moderate change was observed. It can be seen from the graph that the final free amino content decreases with increasing pH, i.e. the degree of the conjugation increases with rising pH, which is in accord with the SDS-PAGE results. The highest conjugation degree of 40% was achieved at pH 10.5. It should be noted that for RPH without MD samples, a decrease of about 10% in amino residues was also detected (data not shown). As mentioned above, this result is due to a certain Maillard reaction of RPH with its saccharide impurities and is also in accord with SDS-PAGE, and visual results.

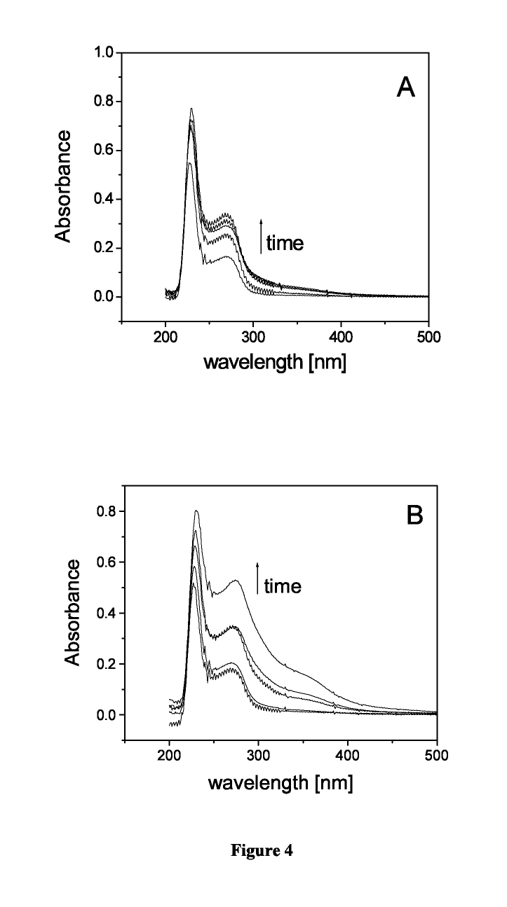

A typical absorbance change with heating time is shown in FIG. 4. The absorbance patterns of RPH with and without MD are compared. At time 0 the typical protein peaks at 230 and 280 nm were observed. Generally, for both RPH and RPH-MD samples an absorbance increase in the UV region was observed, with significantly higher extent for RPH-MD. Especially, the peak increase at 280 and appearance of peak at 350 nm is much more pronounced for the RPH-MD samples. The absorbance increase at 350 nm together with the increase above 400 nm are responsible for the browning of compounds undergoing the Maillard reaction. It is evident that the 280 nm peak growth is also correlated with the Maillard process and it corresponds to the early stage Maillard products (Schiff base).

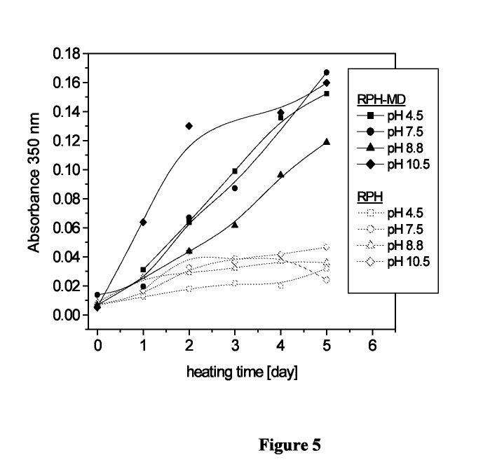

FIG. 5 shows the change of absorbance at 350 as a function of heating time of RPH-MD or RPH samples. Based on FIG. 4, the absorbance value at 350 nm is a good indicator for RPH MD Maillard conjugation process. It can be seen from FIG. 5 that the absorbance rises significantly with heating time for the RPH-MD samples. Similar curves are observed for pH 4.5 and 7.5. Surprisingly a lower slope for pH 8.8 is observed. A steeper rise was observed for pH 10.5. However, the SDS-PAGE analysis showed a more dramatic effect for pH 10.5. It should be mentioned that different reaction routes are involved for different pH conditions, meaning the Maillard products might be different. As a result the reaction rate comparison for different pH cannot be performed according to the absorbance only, since compound of diverse extinction coefficients are produced.

To summarize the above results it can be determined that among the pH values studied, the Maillard reaction is quickest and most efficient at pH 10.5. However, for food applications, processing at extreme alkali conditions is not recommended.⁶⁰ Moreover, pH 10.5 is less preferable in Maillard process due to high extent of melanoidins formation. For samples at initial pH of 4.5, 7.5 or 8.8 after 4 days of heating the conjugation similarly progressed to a significant extent. Based on the above considerations, subsequent experiments focused on the RPH-MD samples obtained after 4 days of heating at initial pH of 7.5.

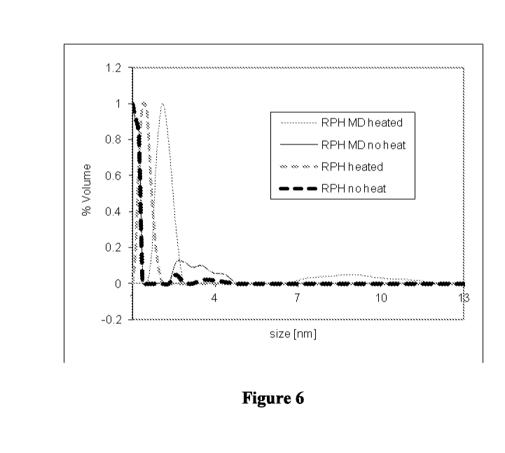

FIG. 6 shows the volume-weighted distribution of RPH-MD particle diameters determined by DLS. The size distribution of heat treated RPH MD (conjugate (conj)) sample can be compared to the control samples of unheated RPH MD (mixture (mix)), as well as heated and unheated RPH alone. It was observed from the figure, that for all samples the main peak of the particle size distribution is of a few nanometers. For the unheated samples of RPH or RPH-MD the obtained mean diameter is 1 nm. Notably, the DLS measurement is at the edge of its sensitivity at such low sizes, thus the obtained value is approximate. However, it can be estimated that the unheated samples give particle sizes lower than the heat treated RPH and the size of latter is smaller than that of the heat treated RPH-MD. It can be seen that the Maillard reaction of RPH and MD created larger particle diameter (2.1 nm) which is another indication of the conjugation, and possibly also an indication of some self-assembly of the conjugates into micellar nanoaggregates. According to previous results RPH in the absence of MD undergoes Maillard reaction with saccharide impurities which results in a certain particle enlargement too.

Interaction of RPH-MD with CLA or VD

Next, the functionality of the conjugates for nanoencapsulation of model hydrophobic nutraceuticals (HN), conjugated linoleic acid (CLA), and vitamin D (VD) was investigated by DLS and turbidity measurements. It was hypothesized that, the co-assembly of HN and the amphiphilic conjugates should result in the formation of smaller colloidal particles, compared to aggregates of the HN alone dispersed in water, consequently leading to formation of more transparent solutions. Moreover, it was hypothesized that the encapsulation of the HN by a “shell” of the conjugates would provide some protection against degradation. The RPH itself might serve as such amphiphile and further improvement of its amphiphilic and protective properties could be achieved by the Maillard based block-copolymers.

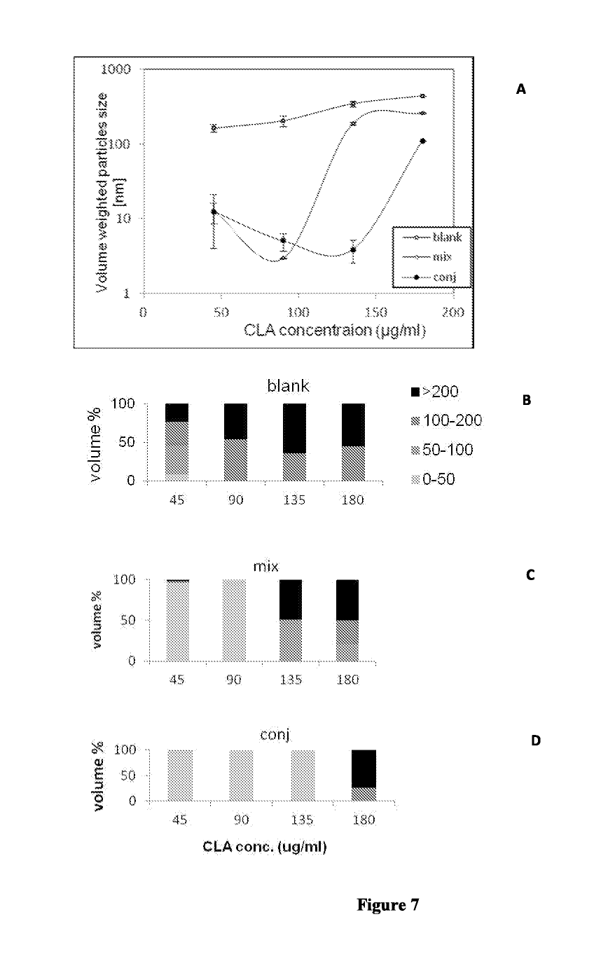

FIG. 7 shows the particle size distribution of CLA as a function of concentration alone and in the presence of conjugated and unconjugated RPH and MD. It was observed that CLA in a buffer without the protein forms colloidal particles of a few hundred nanometers and their size increases with CLA concentration. When the protein was present in the samples it caused a decrease in the CLA particle size which proves that the interaction with the protein improves the dispersibility of CLA. According to the graph, below 100 μg/ml CLA both RPH-MD mixture and RPH-MD conjugate cause a similar decrease in particle size. At 135 μg/ml CLA the conjugate has an advantage over the RPH-MD mixture, meaning that the RPH-MD conjugated exhibits better properties for CLA entrapment. A more detailed particle size distribution can be seen in FIGS. 7 (B), (C) and (D) which presents the volume weighted size fraction at intervals of 0-50, 50-100, 100-200, and >200 nm. It can be seen that in the CLA concentration range of 0.05-0.015 μg/ml all the RPH-MD-based particles are 0-50 nm. For the blank samples there are fractions of particles at higher diameters (50-100, 100-200, and >200 nm) that grow with increasing CLA concentration. CLA dispersed in the protein containing samples produced significantly smaller particles which reduced the turbidity of the colloid solution. It can therefore be concluded that addition of CLA to a buffer containing RPH and MD significantly decreased the particle size of the solution.

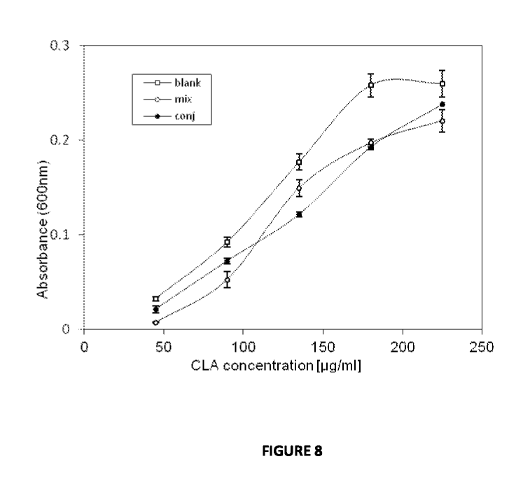

This phenomenon is further depicted in FIG. 8 wherein absorbance at 600 nm was measured as a function of CLA concentration in different sample solutions. It was observed that CLA showed lower absorbance values in a protein-containing solution than without any protein, however, the difference between the conjugate and the mix results was not significant. In other words CLA in the RPH-MD conjugate and mix formed a more transparent solution than control (no protein), but the mix and the conjugate produced similar results.

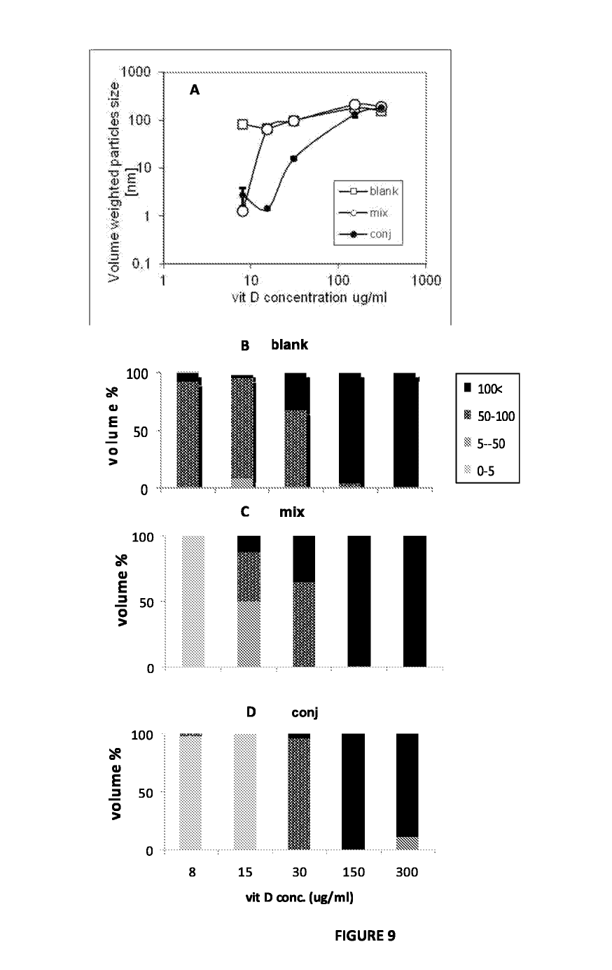

As mentioned above, VD was another model HN studied. VD particle sizes with and without RPH and MD mix or conjugate, at a concentration range of 10 to 300 μg/ml are shown in FIG. 9. It can be seen that at VD concentrations between 15-150 μg/ml in the presence of the conjugate the obtained particles were significantly smaller than in the blank sample. The RPH-MD mixture caused VD particle size reduction only at 10 μg/ml. At higher concentrations the mix gives similar results to the blank samples. A more detailed study of a particles size distribution is shown in FIGS. 9 (B), (C) and (D) which presents the volume weighted size fraction at intervals of 0-5, 5-50, 50-100 and >100 nm.

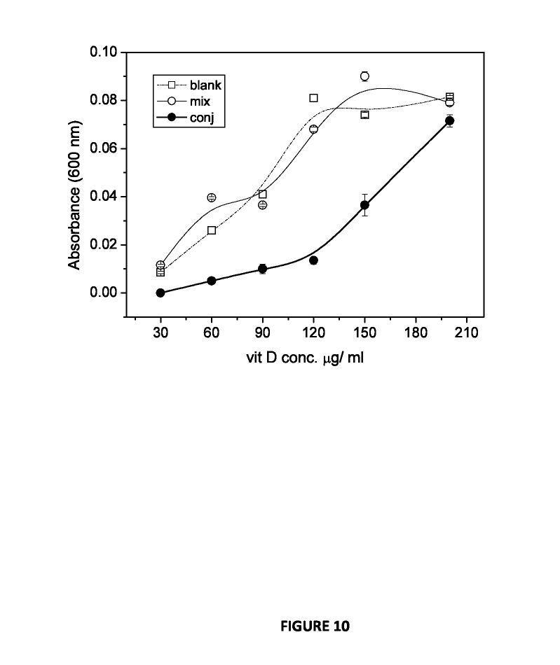

The turbidity test shown in FIG. 10 complements the VD particle size results. In accordance with these results, in the presence of the conjugate the absorbance values were lower than those obtained for the mix or blank samples. Similarly to particle size results the mix and blank samples gave close values in terms of absorbance at 600 nm.

### Ultra-High Pressure Homogenization Process for Particle Size Reduction

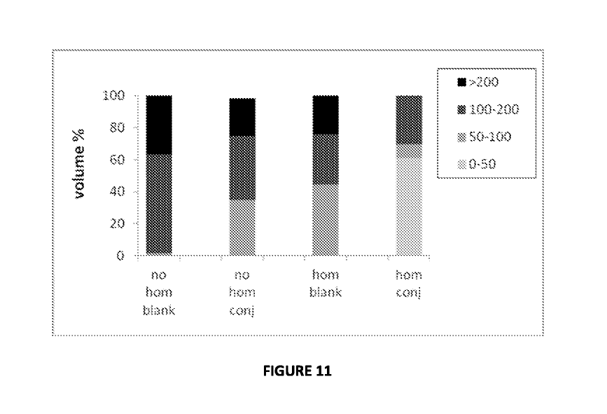

To further improve clarity and homogeneity, an ultra-high pressure homogenization process was employed on the samples of VD with and without the conjugate. In FIG. 11 the influence of homogenization on VD particle size distribution is demonstrated. It was observed that the main fraction of particles in the blank sample was between 100-200 nm. In the presence of the RPH-MD conjugate a smaller fraction of 50-100 nm appeared as expected due to the co-assembly with the amphiphilic conjugates. Further particle size reduction was observed for samples with RPH-MD that underwent homogenization, resulting in a major particle size fraction at 0-50 nm. These experiments show that, in order to obtain smaller particles and even better transparency (where transparency is insufficient), a homogenization process can be considered.

### Protection of CLA and VD During Simulated Shelf Life

After the interaction between RPH-MD and HN was evidenced by DLS and turbidity measurements, the protective capability of the conjugate nanovehicles was evaluated. The CLA and VD are known to be unstable if exposed to oxygen and/or acidic conditions. The entrapment within the RPH-MD nanocapsules was hypothesized to provide some protection to CLA and VD against degradation processes during product shelf life. Without wishing to be bound by any particular mechanism or theory, it is hypothesized that the protection capabilities of the proposed system are not only due to immobilization and physical shielding of the HN by the conjugates, but also due to antioxidant features of the conjugates and their building blocks and Maillard reaction by-products. It was recently shown that the hydrolyzed proteins possess antioxidant features. Maillard products were also shown to serve as antioxidants.

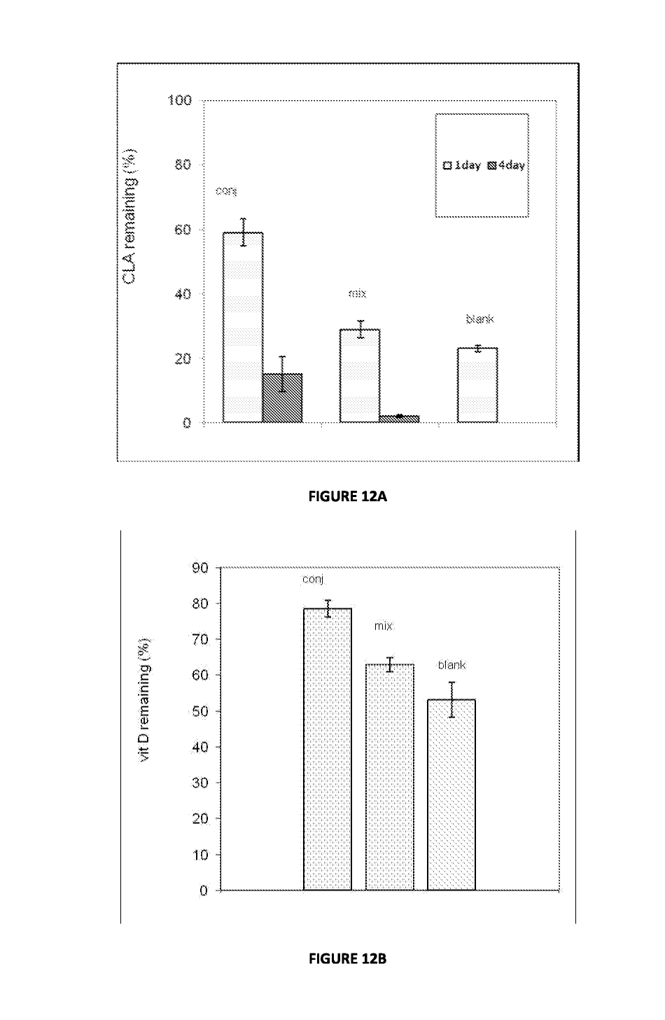

FIGS. 12A and 12B show, respectively, the degradation of CLA and VD under acidic conditions are presented. FIG. 12A shows that the residual percentage of CLA in the blank samples was about 20% and 0% after 1 day and 4 days, respectively. Only a minor improvement in residual CLA was achieved in the presence of RPH-MD mixture. However, about a 2 fold improvement was achieved in conjugated RPH-MD samples compared to the mix after 1 day. After 4 days 15% CLA remained in the presence of RPH-MD conjugate (about 7 fold better than the mix). The results show that the RPH-MD conjugate provides significantly better protection to CLA compared to the RPH-MD mix or the blank sample.

The protection of VD against a 2 hr acidic pH treatment is shown in FIG. 12B. The residual VD was 50, 60 and 80% in the blank, mix and conjugate samples, respectively.

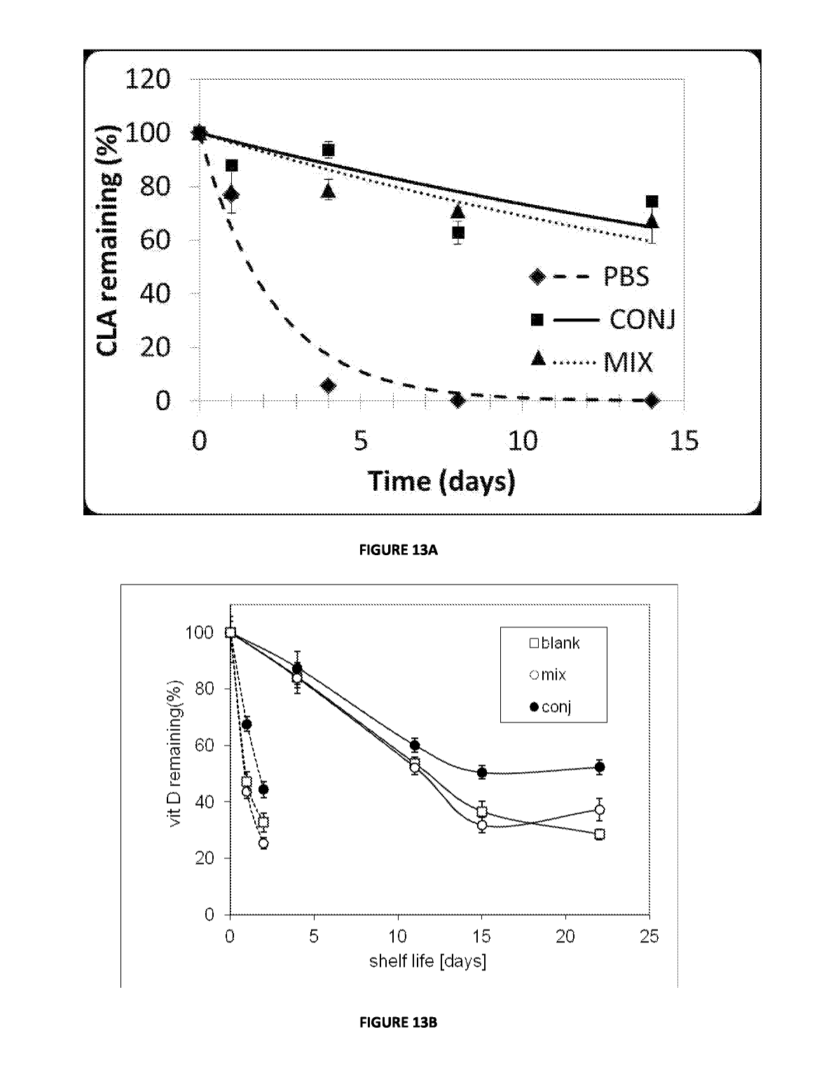

FIG. 13A shows the residual CLA percentage at RT over a period of 14 days in blank, mix and conjugate samples is displayed. It was observed that a steep drop in CLA percentage occurred in the unprotected blank samples, which reached a negligible level after 4 days of shelf life. In contrast, significant protection was obtained by the RPH with a small advantage to the conjugate over the mix samples.

Next, the shelf life of VD was examined at RT and at 4° C. in blank, mix and conjugate samples and the results were summarized in FIG. 13B. A steep drop in VD concentration was observed at RT, with fastest decline in the blank, and slowest in the conjugate protected sample. An expectedly better preservation was obtained at 4° C., presumably since oxidation processes are slowed down with decreasing temperature. At both temperatures the mix samples gave similar results to the blank samples. In contrast, approximately a 2 fold enhancement in residual VD was achieved in the presence of the conjugate at the final time point of each experiment. These results indicate that the conjugate provides better protection to VD compared to the mix, although additional protective means might be needed. It should be borne in mind that these experiments were performed in relatively harsh conditions in terms of oxygen content, while in carbonated drinks oxygen level is much lower due to the deaeration effect of carbonation.

### Conclusions

### Example 1

The Maillard reaction of RPH and MD was examined by SDS-PAGE analysis as a function of time at different pH conditions. For pH 4.5, 7.5 and 8.8, similar progression was observed showing gradual Mw increase of the oligo-peptides species due to covalent bonding with the MD molecules. For pH 10.5, after one day of heating a significant Mw increase was obtained i.e. the reaction rate was the highest at this pH. These results were confirmed by color development studies. The absorbance measurement showed an increase as a function of reaction time at spectral region typical to Millard reaction products. The free amino residues concentration decreased with the Maillard reaction progression as was shown by the OPA analysis. The highest decrease was obtained for pH 10.5 samples in agreement with other methods of analysis. The amino residues content decreased mainly on the first day of heating and then stayed approximately unchanged during the rest of the time.

The size distribution of the obtained RPH-MD conjugates was measured by DLS and an enlargement of particle size compared to the control samples was shown which served an additional proof for the conjugation process, and a possible clue for some self-assembly.

The interaction of the RPH-MD with two model HN: CLA and VD, was studied by DLS and turbidity analyses. Significant reduction of the HN particle size in the presence of RPH-MD was achieved, which also resulted in a decrease in turbidity of the mixture (i.e., increased transparency). These effects were attributed to the solubilization capability of the RPH-MD due to co-assembly with the HN. RPH-MD conjugates were found to be more effective dispersants than the RPH-MD mixture, i.e. the conjugation has improved the RPH amphiphilic properties.

The protection of VD and CLA during the shelf life by the RPH-MD mix and conjugate was evaluated. It was found that both model HN compounds underwent significant degradation under acidic conditions but significant improvement was achieved in presence of RPH-MD with considerable advantage of the conjugate over the mix. Examination of CLA shelf life at neutral pH and RT showed significant improvement of the residual percentage of CLA in the presence of the RPH-MD conjugates. After 14 days it was 0, 70 and 75% for blank, mix or conjugate samples, respectively. For VD after 22 days of the simulated shelf life at neutral pH and 4° C., 28, 37 and 52% of residual VD were obtained for blank, mix or conjugate samples, respectively. These results demonstrate the good potential of the HRP-MD Maillard conjugates (and Maillard conjugates in general) as natural nanoencapsulating materials for HN for application in clear drinks.

### Example 2

### Soy Protein (Beta Conglycinin)-Maltodextrin Maillard Conjugates Based Nanoparticles for Protection of Nutraceuticals

The objective of this part of the study was to form Maillard conjugates of soy proteins and maltodextrin, characterize them and evaluate their potential for nanoencapsulation of nutraceuticals, preferably for clear liquid systems.

### Materials

Soy protein beta-conglycinin was kindly donated by Solbar (Solbar Plant Extracts Ltd, Ashdod, Israel). Beta conglycinin was chosen as it is more amphiphilic than the other major soy protein, glycinin. It was dialyzed against distilled water with Spectra/Por molecular porous membrane tubing (M.W.C.O 12-14 kDa). However, it is apparent to a person of skill in the art that other soy proteins including but not limited to glycinin can also be used to form the Maillard conjugates of the present invention.

(−)-Epigallocatechin-3-gallate (EGCG) (CAS registry number 989-51-5) (EG-090, purity>90% by HPLC) EGCG was chosen as the model hydrophilic nutraceutical compound for this study, because of its highly important health benefits, and the challenge of protecting water-soluble nutraceuticals. Additionally, it is a good model for nutraceutical study, as it changes its color upon oxidative degradation. It was purchased from Shanghai Angoal Chemical Co. (Shanghai, China).

### Methods

### Preparation of Conjugates

Freeze dried solutions of beta-conglycinin (β-cong) and Maltodextrin (MD) DE=19 at molar ratios of 1:1, 1:2, 1:4, 1:8 were heated (60° C. at 79% RH) for 6 hrs to form conjugates by Maillard reaction. After heating, the samples were freeze dried. The Maillard reaction products were dissolved in phosphate buffer solution (PBS) pH 6.87, 30 mM.

### Water Solubility

Conjugate solution (β-cong: MD molar ratio 1:8), mixture solution (β-cong: MD molar ratio 1:8) and β-cong solution were centrifuged at 15700′ g for 1 min. B-cong concentration was 0.25% w/v in all three solutions. The pellet was dried overnight in an oven at 100° C. and then weighted by an analytical balance. The percentage of soluble material was calculated as:

$$\% soluble\_material = \frac{C \times V - Wp}{C \times V} \times 100 \%$$

C=the conjugate/mixture/β-cong concentration
V=the solution volume
Wp=the pellet weight

### Determination of Particle Size Distribution by DLS

Particle size distribution was determined by a dynamic light scattering (DLS) analyzer (NICOMP™ 380, Agilent Technologies, Inc., Santa Barbara, Calif., USA) equipped with an Avalanche Photo Diode (APD) detector, used at a fixed angle θ=90°. The 90 mW laser wavelength was 658 nm. Mono- bi- or tri-modal distributions were calculated from the scattered light intensity fluctuations, by Nicomp™ cumulants analysis of the autocorrelation function. Measurements were made in duplicate at 23° C.

### Evaluation of the Protection of EGCG by Maillard Conjugates, Using Visible Spectrophotometry

Conjugate solution (β-cong: MD molar ratio 1:8), mix solution (β-cong: MD molar ratio 1:8) and β-cong solution were centrifuged at 15700′ g for 1 min, and the supernatant was collected. EGCG solution (0.125% w/v 30 mM PBS, pH 2.5) was added to the supernatant. The final EGCG concentration was 0.0125% w/v. The final con/mix/β-cong concentration was 0.092% w/v and the pH of the final solution was 6.69. After adding the EGCG solution, the samples were vortexed for 20 seconds.

After preparation, the samples were placed in 1 cm path length spectrophotometer cuvettes and covered with parafilm. Absorbance at 425 nm was recorded with time at room temperature for 300 hrs.

### Results and Discussion

### Maillard Conjugate Formation: Analysis by DLS, and Solubility Measurements

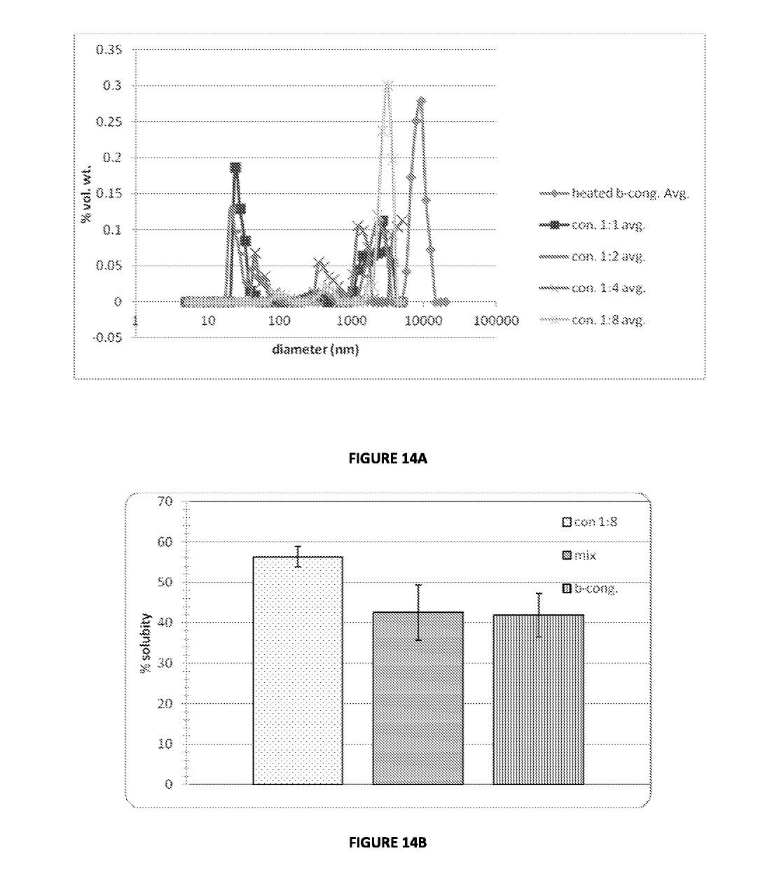

The Maillard reaction products at different molar ratios (β-cong: MD) were dissolved in PBS pH 6.87, 30 mM, and particle size distribution was measured by DLS. The results are shown in FIG. 14A. It can be seen that in PBS the heated n-cong, which was subjected to the same heat treatment as the conjugates (60° C. at 79% RH for 6 hrs), formed large particles of around 10,000 nm in diameter. The conjugates formed two or three particle populations—large particles of more than 1000 nm in diameter, and small particles of less than 100 nm. The large particle population is suggested to be due to aggregation during the heat treatment. B-cong has one cysteine group at its α and α′ subunit, which can initiate SH—SS interchange-chain reaction, resulting in aggregation.³³ The small particle population might be due to conjugation, as the MD covalently bonded to the protein and due to steric hindrance inhibited protein aggregation. As the β-cong: MD ratio increased, the mode particle size of the small particle population increased, which supports the assumption that this population comprises conjugates.

To separate the two populations, the samples were centrifuged at 15700′ g (13000 rpm) for 1 min. After centrifugation, the pellets were dried overnight in an oven at 100° C., and then weighted by analytical scales. The percentage of soluble material was calculated as described in the Methods section; results are shown in FIG. 14B.

Mix solution was obtained by adding MD solution to β-cong solution, at the same b-cong:MD molar ratio as that of the conjugate. MD and β-cong were subjected to the same heat treatment as the conjugates (60° C. at 79% RH for 6 hrs).

As seen from FIG. 14B, the conjugate shows higher solubility in aqueous solution compared to the mix and the β-cong. There is no significant difference in solubility between the mix and the β-cong alone. The conjugation process therefore improves protein solubility in aqueous solution, which is an advantage for nanoencapsulation applications.

### Nanoencapsulation of EGCG as a Model Nutraceutical Substance, in β-Cong-MD Conjugates

(−)-epigallocatechin-3-gallate (EGCG) was chosen as an oxidation-sensitive bioactive. EGCG is one of the main effective constituents of green tea; it is a water soluble polyphenol which is highly unstable in neutral and alkaline solutions.

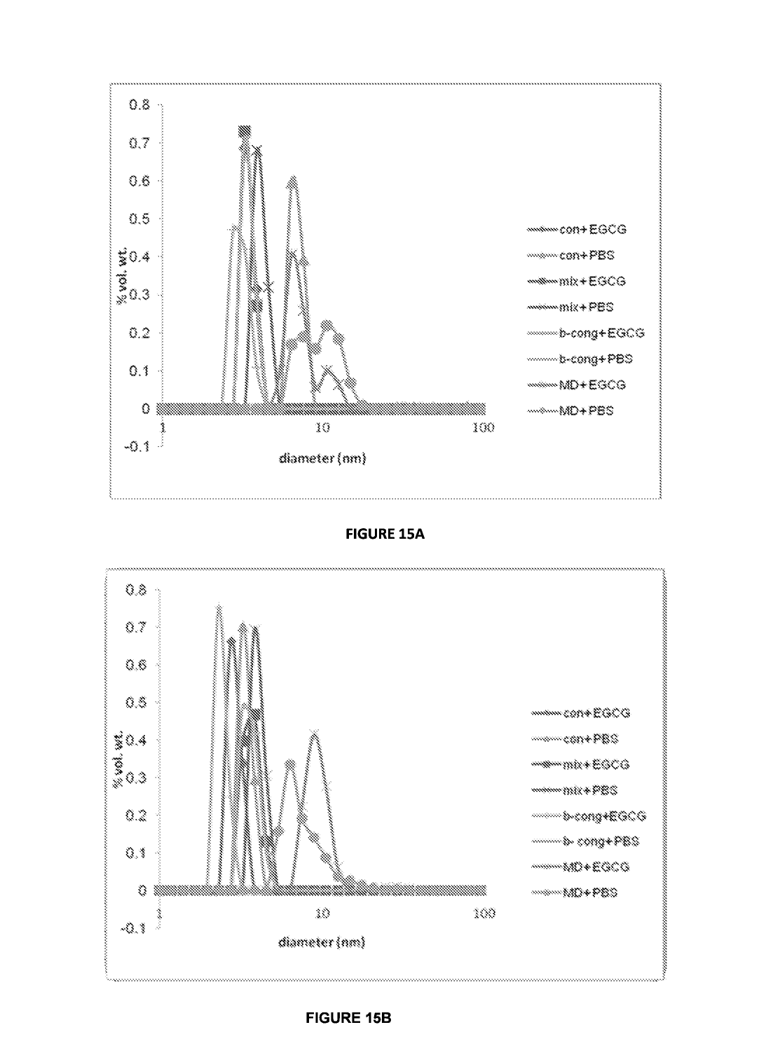

The co-assembly of the conjugates with EGCG was studied by measuring particle sizes. The changes in size distribution of the system were also measured after 48 hrs. Results are shown in FIGS. 15A-B. It was observed that all particles are smaller than 20 nm, an advantage for clear systems. The conjugate peak at time zero without EGCG is intermediate in size between MD and β-cong alone, supporting that conjugation occurred. The conjugates, with and without EGCG gave smaller particle sizes than solutions of MD with and without EGCG. These results suggest that the conjugation facilitates formation of smaller-more soluble entities. After 48 hrs, particle sizes remained rather small, still below 20 nm.

### Protection Conferred to a Model Nutraceutical Substance by the Conjugates

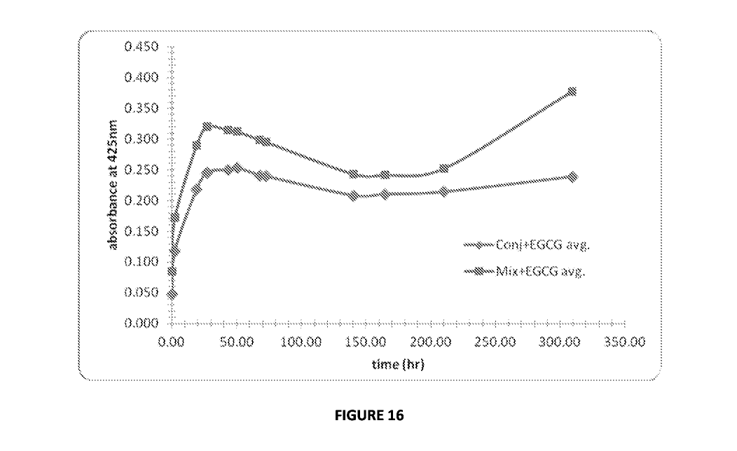

EGCG degrades irreversibly from a colorless clear solution of the fresh compound, to a yellow solution of the deterioration products, mainly due to oxidation and the formation of dimers. To estimate the protection against oxidation provided by the conjugate, the absorbance of EGCG with the conjugate was measured at 425 nm. Protection provided by control sample of the mixture solution of β-cong and MD was also monitored. Results are shown in FIG. 16. It can be seen that the protection provided by the conjugate is more significant than the protection provided by the mixture control sample.

### Conclusions

### Example 2

These results of this study emphasize the potential of soy β-cong-MD Maillard conjugates as nanoencapsulation material for clear drink applications. The conjugates showed better solubility than the mixture of their components. The conjugates, with and without EGCG gave smaller particle sizes than solutions of MD with and without EGCG. The conjugation apparently facilitated formation of smaller-more soluble entities, with particle sizes that remained below 20 nm after at least 48 hrs from preparation. The protection provided by the conjugate-based nanoparticles to EGCG was more significant than the protection provided by the mixture control sample.

### II. Nanocapsules Made of Maillard Reaction Based Conjugates of Milk Proteins and Maltodextrin

### Example 3

### Casein and Maltodextrin Based Nano Vehicles for Nutraceutical Delivery

The objective of this part of the study was to form Maillard conjugates of casein and maltodextrin (MD), characterize them and evaluate their potential for nanoencapsulation of hydrophobic nutraceuticals, preferably for clear liquid systems. The behavior of the nanocapsules during simulated gastric digestion was also studied.

### Materials

Maltodextrin (MD) of dextrose equivalent 19 which corresponds to approximately 10 KDa was donated by Productos de maiz S.A. Corn Products international (Munro, Argentina). Caseinate was donated by Strauss-group, and was manufactured by Molkerei Meggle Wasserburg GmbH and co. (Casinella QN lot number 901155). O-phthaldialdehyde (OPA), Trizma Base, SDS, Vitamin D3 (VD3), Vitamin D2 (VD2), mercaptoethanol, Acrylamide/bis-Acrylamide, Ammonium persulphate, pepsin from porcine gastric mucosa (3200-4500 units/mg), Nile red, were obtained from Sigma-Aldrich (Rehovot, Israel). Methanol and Acetonitrile both of HPLC grade were obtained from LabScan (Dublin, Ireland). NaOH was obtained from Merck (Darmstadt, Germany). Ethanol absolute—BioLab (Jerusalem, Israel), Sodium Tetraborate-Laba Chemie (Mumbai, India). SDS-PAGE size markers and Coomassie Blue 250-R stain were obtained from Bio-Rad. Bromophenol blue was obtained from Fluka.

Epigallocatechin-3-gallate (EGCG) (CAS registry number 989-51-5) (EG-090, purity>90% by HPLC) was purchased from Shanghai Angoal Chemical Co., Ltd. (Shanghai, China).

Acetone was purchased from Frutarom, Israel.

### Methods

### Conjugation Process by Maillard Reaction

Caseinate powder was dissolved in Doubly Deionized water with 0.02% (w/w) sodium azide over-night. Later it was dialyzed for 48 hrs, frozen and freeze-dried. Freeze dried solutions of Caseinate and MD at different molar ratios were heated (60° C. at 79% RH) for 4, 6, and 8 hrs. (Similar ranges of conditions were used^{28, 64-67} to form conjugates without significant progression of the Maillard reaction to degradation and/or polymerization). After heating the conjugates were freeze-dried again.

All characterization procedures were made on both CN-MD conjugates (conjugates) and CN with MD mixture (mixture). Mixture control-samples of casein (CN) and MD were heated separately, than mixed.

### SDS-PAGE Analysis

The Maillard reaction was tracked by electrophoresis method using PHAST system (Pharmacia LKB Biotechnology, GE Healthcare), PhastGel gradient 8-25, and PhastGel SDS Buffer Strips, both manufactured by GE Healthcare were used. The samples in a form of lyophilized powder were dissolved in a sample buffer (50 mM Tris, 1% SDS, 2.5% mercaptoethanol, 10% glycerol, 1 mM EDTA, 0.025% bromophenol blue). The samples were incubated for 5 min at 95° C. with vigorous stirring. A final sample concentration of 5 mg/ml on protein basis was obtained. A volume of 1 μl was loaded on the gel. After the electrophoresis the gels were immersed in a fixation solution (30% methanol, 10% acetic acid) for 0.5 hr, then stained in a Coomassie Brilliant Blue R-250 for 1 hr and washed by 10% acetic acid solution.

### Estimation of Conjugation Degree by OPA Assay

The RPH-MD graft samples were analyzed by the OPA assay to determine the degree of conjugation. The OPA reagent was prepared as described above in Example 1. The following compounds were diluted with water to 100 ml: 80 mg OPA (dissolved in 2 ml 95% ethanol); 50 ml 0.1 M sodium tetraborate, 5 ml 20% SDS; 0.2 ml of 2-mercaptoethanol in ethanol. The OPA reagent was prepared immediately before use. The CN-MD conjugates and mixture samples at concentration of 0.5 mg/ml on casein base were prepared in DDW with 0.02% azide. 0.05 ml of the sample was added to 2 ml of OPA reagent. This solution was briefly stirred and absorption at 340 nm was measured after a 2-min equilibration at room temperature. A standard curve was obtained by using L-leucine as a reference compound. Reference samples with a concentration ranging from 1.52E-5 to 7.62E-3 M were prepared in DDW 0.02% sodium azide and the L-leucine determination was performed as described above.

### Isoelectric Precipitation

The samples at concentration of 1 mg/ml were dissolved in ddw, and acidified to pH=4.6 (pI of casein), followed by separation of the precipitate by centrifugation at 1000 g for 10 minutes. Then, the supernatant was transferred to a new tube and pH was adjusted back to 7 with NaOH 5 M. The protein content of the pre-separated solution and supernatant were measured by absorbance at 278 nm. The yield of separation was defined as follows:

$$yield ≡ \frac{supernatant absorbance ( 278 nm )}{pre \text{-} separated conjugate solution absorbance ( 278 nm )}$$

Absorbance was measured by Ultrospec 3000 UV/Visible Spectrophotometer, GE healthcare.

### Particle Size Distribution Analysis by Dynamic Light Scattering (DLS)

The particle size evaluation was performed by dynamic light scattering (DLS) analyzer (NICOMP_{—}380, Particle Sizing Systems Inc., Santa Barbara, Calif., USA). The detector angle was set to 90 degrees. Samples of mixture, conjugates, and supernatant with and without HN were analyzed. ND (“neutral density”) filter (light intensity adjustment, which is an indication of the amount of scattered light from the sample) was kept in the range of 70-120, by adjusting sample concentration, to avoid multiple scattering.

### Addition of VD to CN-MD Solutions, and Forming Nanocapsules.

The co-assembly of the HN with the conjugates was achieved by addition of the VD or Nile red (NR) dissolved in ethanol into the CN-MD solution during stirring. The HN ethanol solutions were prepared at different concentrations while the final ethanol concentration was kept constant 0.25% (vol/vol). All solutions containing VD were flushed with argon gas to prevent oxidation.

### Examination of VD Preliminary Extractions

A. Extraction of VD using phase separation in a separatory funnel of diethyl ether: petroleum-ether was accomplished as was previously described.³⁶ While this procedure was good enough for extracting the VD from a mixture solution, it was insufficient for extracting it from the conjugates, as they stabilized an emulsion and no phase separation occurred, preventing extraction (an indication of the superior encapsulation capacity of the conjugates compared to that of the mix).

B. Extraction of VD using a method based on Kazmi et al:³⁷ 1 ml of sample solution was put in a glass centrifuge tube, 3.5 ml of methanol:chloroform (2:1 vol/vol) were added, then the tube was vortexed for 20 seconds, followed by addition of 1.5 ml chloroform and vortexing for 60 seconds. Argon gas was added to the headspace and the tubes were capped and centrifuged for 10 minutes at 1500 g at 4° C. Two ml of the clear chloroform layer at the bottom of each test tube were transferred to an evaporation vial using a glass syringe. The chloroform extract was dried under a flow of nitrogen gas, reconstituted in 1 mL of the high performance liquid chromatography (HPLC) mobile phase [methanol:acetonitrile:water (49.5:49.5:1 v/v)], the tube headspace was filled with argon gas. The tubes were left undisturbed for 15 min, after which the samples were put on ice until injection to HPLC. Operating conditions were: ambient temperature (˜24° C.); mobile phase was methanol: acetonitrile: water (49.5:49.5:1, by vol); flow rate was 0.3 ml/min; and the absorbance was measured at 265, 254 and 228 nm.

C. VD degradation at pH=2.5: VD2 and VD3 in buffer solutions were made by addition of VD stock solution in ethanol into phosphoric acid buffer at pH=2.5, to a final concentration of 0.05 mg/ml. Samples were incubated at room temperature for 2 hrs and then analyzed for VD. Peak areas were compared to calibration curves.

### VD2 and VD3 Protection During Shelf Life.

The protection of VD2 by the conjugates as a function of time, compared to controls of mixture and buffer was evaluated. Simulated shelf life conditions of temperature 4° C. and pH 7 were tested. For pH 7 a NaH₂PO₄/Na₂HPO₄ buffer was used. Samples were flushed with argon, heated for 2 minutes at 80° C., and kept at 4° C. for 15 days. VD was analyzed, before heating, after heating and after 1, 3, 5, 9, 13, and 15 days. Extraction was carried out, then the VD content was quantified using reversed phase HPLC (RP-HPLC), equipped with 4.6×100 mm C18-C2 Pharmacia column and a triple wavelength UV detector. The volume of the injection loop was 100 μl. The operation temperature was 24° C. The initial concentration of VD2 was 0.05 mg/ml (simulating a concentrate of an enriched soft drink). The molar ratio of VD to CN was 1:1.

### EGCG Protection During Shelf Life

EGCG was chosen as a model for a sensitive water soluble nutraceutical. It was dissolved in phosphoric acid buffer (20 mM) pH=2.5, and added to solutions of the conjugate, and the controls: CN: MD mixture, CN, MD, PBS (pH=7) and phosphoric acid buffer (pH=2.5). In mixture and conjugate solutions casein concentration was 5 mg/ml, MD: CN molar ratio was 4. In casein and MD solutions each substance concentration was the same as in the mixture and the conjugate. EGCG concentration was 0.9 mg/ml. Samples were kept at room temperature.

EGCG oxidation was measured by determining absorbance at 425 nm, based on the observation that EGCG oxidation products absorb at 425 nm.

### Use of Nile Red as a Model for Hydrophobic Nutraceuticals

Nile read (NR) was chosen as a model for a hydrophobic nutraceutical, as it is similar in structure and properties (Table 1) to such hydrophobic nutraceuticals like VD, and it can be easily determined by spectrophotometry, and spectrofluorometry. Moreover, it “reports” of its binding or hydrophobic entrapment, by changing its fluorescence. Nile red is known as a probe for hydrophobic domains and as a probe for protein hydrophobic surfaces. When in water it adsorbs to the glass and does not fluoresce.

The chemical structures of a—Nile red, b—Vitamin D2, c—Vitamin D3 are set forth below:

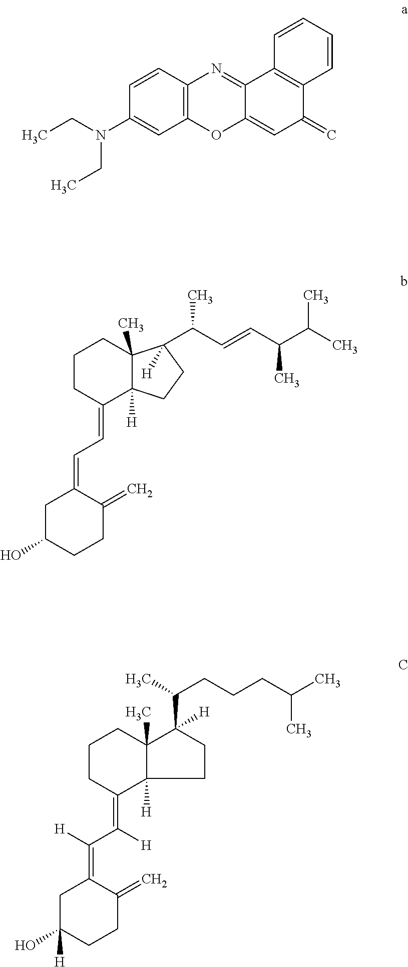

TABLE 1
a comparison between Nile red, VD2, VD3
in terms of logP and molecular weight

| Compound | Calculated Log P | Mw (gr/mol) |
|---|---|---|
| Nile Red (NR) | 5 | 318.37 |
| Vitamin D2 (VD2) | (unavailable, similar to VD3) | 396.67 |
| Vitamin D3 (VD3) | 9.14 | 384.64 |

NR stock solution in ethanol was prepared at a concentration of 0.16 mg/ml=502.6 μM. NR was added during vortexing to the conjugate, mixture or buffer solution, at a concentration of 1.3 μM, to minimize inner filtering effect in fluorescence measurements.

### NR Absorbance Spectra:

NR was added as mentioned above to ethanol, buffer pH=2.5, water, conjugate solution, and mixture solution. Casein concentration was 5 mg/ml, MD: CN Molar ratio was 4. NR final concentration was 1.3 uM. Absorbance was measured in a quartz cuvette (path length=10 mm) using Ultrospec 3000 UV/Visible Spectrophotometer, (GE healthcare).

### NR Emission Spectra:

All fluorescence measurements were done using a Fluorolog 3-22, (Horiba Jobin Yvon, Edison, N.J. USA). Emission spectra was measured at excitation wavelength (ex) of 570 nm, slit width was 5 nm for both excitation and emission. NR final concentration was 1.3 μM emission spectrum was measured in a quartz cuvette sized 10 mm*2 mm at a right angle mode. Blank measurements were also taken and were two orders of magnitude smaller than that of the NR signal.
Evaluating HN Release from Nanocapsules by Examination of NR Adsorption to Glass:
Free NR in aqueous solution adsorbs to the glass walls, which may serve as a convenient way to study its release behavior from nanocapsules. To examine whether NR adsorbs to glass when in water, NR was added to 1 ml of water in a glass vial to a final concentration of 1.3 μM. The vial was left for 2 hrs, then the water was transferred to a new glass vial, and 1 ml of acetone was added to the new vial. At the same time 2 ml of water:acetone (1:1 vol/vol) solution was added to the first vial. After 1 hr both solutions were read for florescence intensity at ex:570, em:645 which is the intensity peak at water:acetone 1:1. Concentration was calculated from the intensity using a NR in water:acetone (1:1) calibration curve.

### Simulated Digestion Using Nile Red:

Since it was validated that all free NR adsorbs to the glass when in water, detection of NR which did not adsorb to the protein (or was released from it after protein digestion) was carried out as follows: NR was added to CN-MD mixture, to conjugate (casein concentration was 3 mg/ml, MD: CN molar ratio was 4) and to buffer (all at pH=2.5 in phosphoric acid buffer 20 mM) to a final concentration of 1.3 μM. Then pepsin was added to some of the samples at a concentration of 0.15 mg/ml, in order to reach pepsin: CN mass ratio of 1:20 according to the method by Mandalari et al.⁷⁹. The samples were left for 2 hrs at 37° C. while gently stirring to simulate gastric digestion. After two hours, protein aggregates sedimented to the bottom of the vials in which pepsin was present. The solution and the aggregates were taken out using a syringe and put into new vials. Into the old vials we added 1 ml of water: acetone solution. After 1 hr the acetone: water solution with NR which had adsorbed to the glass was read for fluorescence intensity (ex:570, em:645). NR fluorescence was measured with time to validate that no bleaching occurred during the procedure (data not shown).

### Results and Discussion

MD and Casein Conjugation Characterization: Conjugation of Casein with MD DE=6

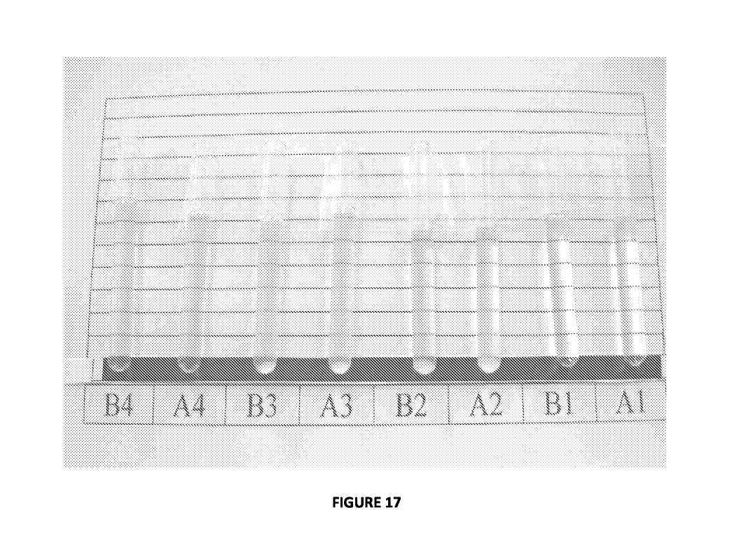

As seen in FIG. 17, MD DE=6 (mw-20 kDa) is insoluble in water at room temperature, therefore the solution containing MD DE=6 was turbid and the MD sedimented. Casein conjugated MD solution was less turbid than both MD and mixture of MD with casein, and the MD did not sediment. It can be inferred that conjugation via Maillard reaction improved the solubility of MD DE=6. The solubility of MD was improved because caseins are much more soluble in water, thanks to their negative charge.

MD DE=6 was not used in later work. Rather, MD with greater solubility was used in order to allow the formation of concentrated homogeneous solutions before conjugation.

Conjugation of Casein with DE=19 MD

### Conjugation Examination Using SDS PAGE.

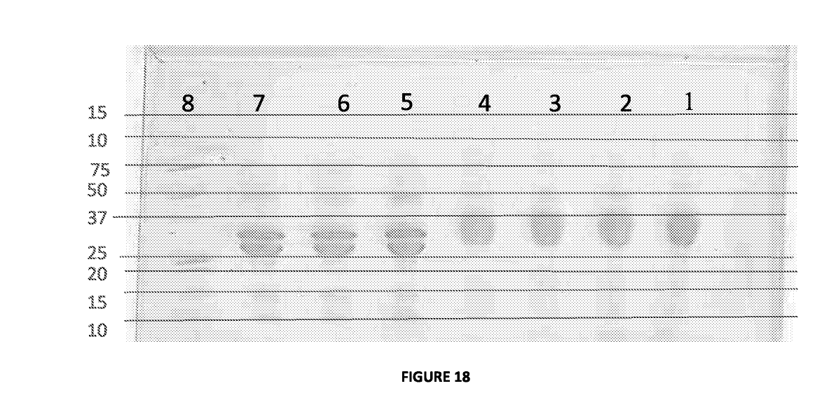

FIG. 18 shows that casein is separated into 3 bands, apparently some of the caseins appear on the same band. After conjugation the whole casein band shifted backward to larger Mw, providing excellent evidence for the formation of Maillard conjugates. The shift of about 5-10 kDa compared to the Casein band (lane 7) suggests of an average conjugation of ˜1 MD molecule per casein (although weak bands of higher Mw appeared too, possibly due to conjugation of several MD to one casein molecule.)

### Conjugation Examination Using OPA Reagent

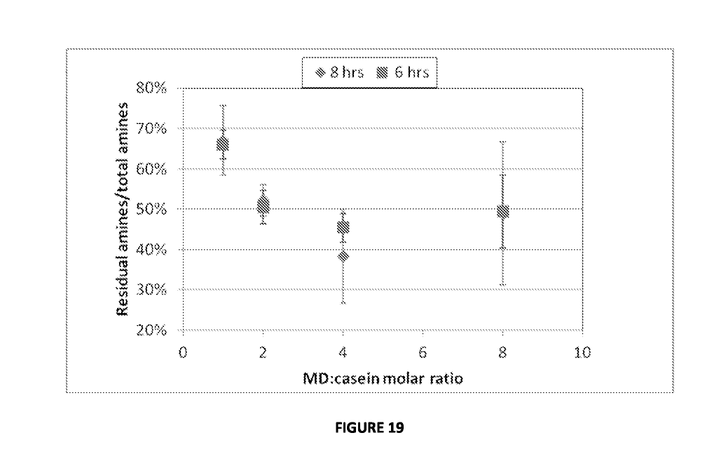

FIG. 19 shows decrease in free amino residues with increasing MD: CN molar ratio, at ratios 1, 2, and 4. As expected, higher MD concentration leads to higher conjugation ratio. At ratio 8 no more amino residues were reacted.

An average casein molecule contains about 13.6 lysine residues. The ratio which resulted in largest decrease in free lysines was 1:4 so theoretically more lysines could have reacted with MD. Without wishing to be bound by any particular mechanism or theory, there are two possible explanations why this did not occur are: 1. Steric hindrance: not all lysines are accessible, and even more so, after one or two MD molecules attached to the casein, the access of additional MD molecules to the casein is even more difficult. 2. During freezing, phase separation occurred and good contact between the MD and the casein was not achieved.

A better conjugation ratio may be achieved by quench-freezing of the mixed solution of MD with casein.

At a ratio of 1:4 it is expected that the maximum decrease in free lysines would be 4/13.6=31%, which means residual amines of about 69%. The observed decrease was of 60%, i.e. more than expected. An explanation for this phenomenon may be that progressive Maillard reactions caused further decrease of free lysines below 69%.

The number of lysine per casein molecule was calculated as follows:

$$lysine per casein [ \frac{{mol}_{lys}}{{mol}_{cas}} ] = ( \frac{{mg}_{lys}}{ml} \text{/} \frac{{mg}_{cas}}{ml} ) \frac{1}{{mw}_{lys}} [ \frac{{mol}_{lys}}{{gr}_{lys}} ] {mw}_{cas} [ \frac{{gr}_{cas}}{{mol}_{cas}} ]$$

### Fractionation of Conjugates by Sedimentation at the Casein pI

Examination of sedimentation at the casein pI

With the aim of fractionating the Maillard products and concentrating the conjugates in a simple, industrially feasible procedure, sedimentation of the unconjugated casein was performed at its pI, and the supernatant containing the conjugates which are still soluble at this pH was collected. The sedimentation at the casein pI, and the yield calculation was performed as described in the Materials and Methods section. The yield calculation was based on spectrophotometric determination of protein concentrations in the supernatant and original solution.

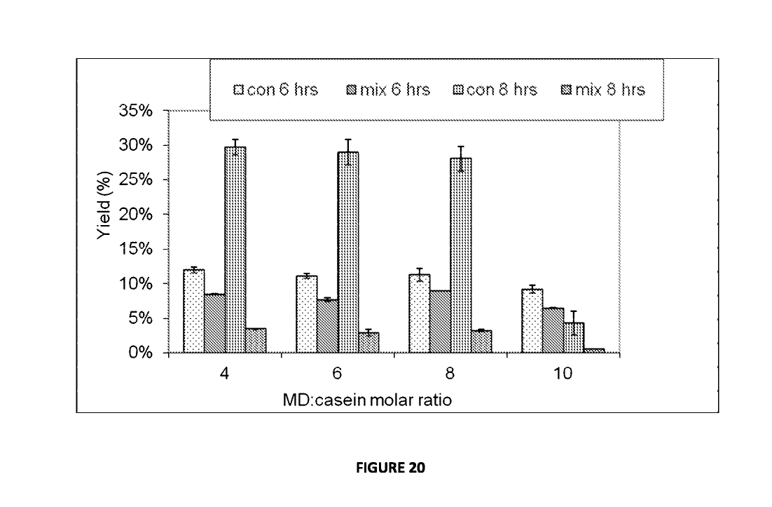

According to FIG. 20, conjugates formed during 8 hrs of heating displayed the greatest yield (about 30%) at MD: CN ratios of 4, 6, and 8. Conjugate of 6 hours had significant lower yields than those obtained in 8 hrs. This may be explained by higher conjugation yield of the Maillard reaction with longer heating time, as its conjugation products are more soluble at the casein pI. Mixture solutions had expectedly significantly lower fractionation yields.

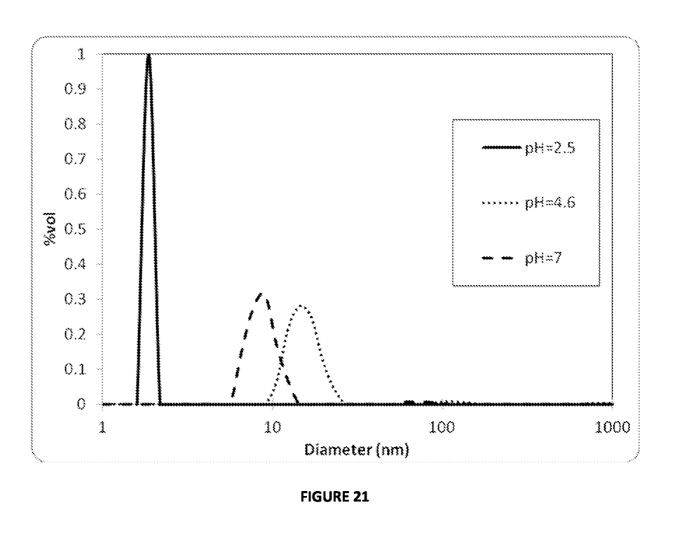

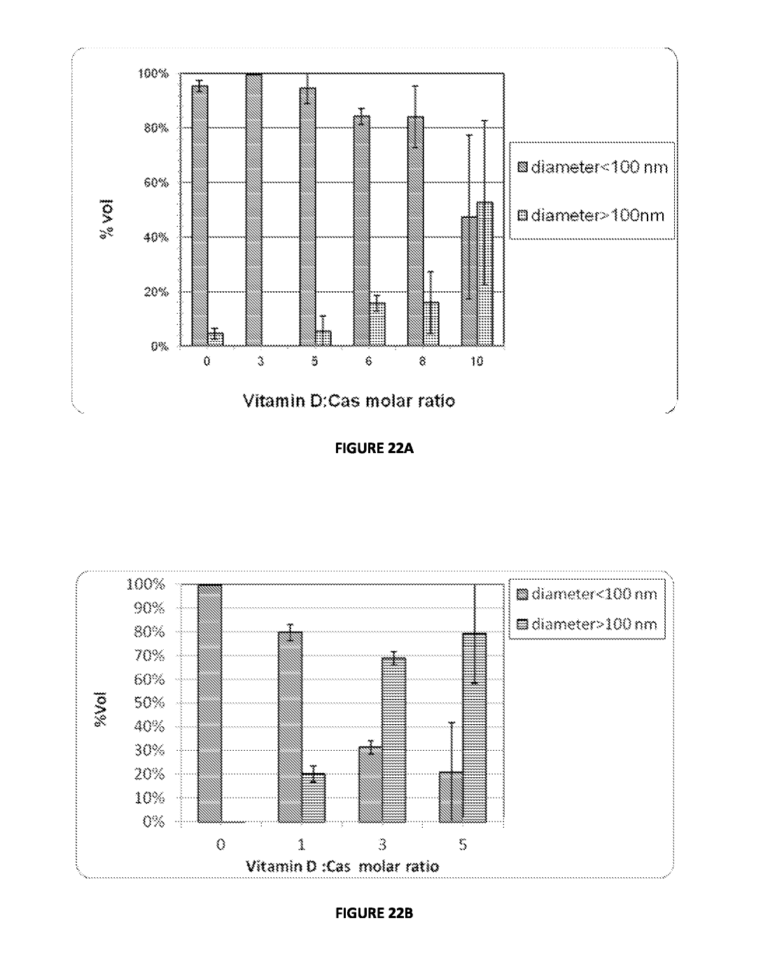

Based on the results presented in FIGS. 21 and 22, further encapsulation procedures were performed with conjugates prepared in 8 hrs of heating, and with a MD: CN molar ratio of 4.

Nanoencapsulation of VD Using pI-Fractionated (Supernatant) Conjugates.

The supernatant was collected; freeze dried and later dissolved in buffers at pH 2.5, 4.6 and 7. VD3, predissolved in ethanol, was added to those solutions during vortexing. The final casein concentration was 1 mg/ml, VD: CN molar ratio was 1:1. The solutions were then measured for size distribution using DLS. Size distribution of particles containing VD with supernatant conjugate at different pH is shown in FIG. 21. As shown, most of supernatant conjugate with VD complexes remain less than 30 nm in diameter even at the casein original pI. The complexes at pH 4.6 were slightly larger than the complexes at pH 2.5 or 7, apparently due to the lower charge, resulting in lower interparticle repulsion. In any case, all nanoparticles formed herein are small enough to enable enrichment of clear drinks with VD. The selection of the pI-soluble fraction resulted in very small nanoparticles formation, even around the Casein pI.

Comparing VD Nanocomplexes Made of CN:MD Conjugates Vs. CN:MD Mixture, and Evaluating the Effect of VD:CN Molar Ratio.

FIGS. 22A and 22B compare CN: MD conjugates vs. CN: MD mixtures respectively, and in each case, study the effect of VD3: CN molar ratio on the particle size distribution. As the concentration of VD rises, the particle size increases. At higher VD concentrations, large particles appear. These larger particles may be either VD aggregates or complexes containing casein and VD.

The range of ratios described in FIG. 22B was only up to VD: CN=5, because at higher VD concentrations the solution was too turbid and could not be read in the DLS. A comparison of FIGS. 22A and 22B shows that particles of the conjugates with VD were smaller than those of CN-MD mixture with VD, and that VD in the conjugate solution scattered less light and was thus much less turbid.

Future studies were conducted with VD: CN molar ratios of 1 and 2 because at those concentrations and ratios mostly particles smaller than 100 nm were formed. At VD concentrations of above 0.05 mg/ml large aggregates were formed. In addition, VD without the conjugates tended to aggregate as expected at all concentrations. Shelf life experiments were conducted for VD at the highest concentration that the conjugate can stabilize and prevent its aggregation, i.e. 0.05 mg/ml. Casein concentration was 3 mg/ml in order to reach 1:1 VD:CN molar ratio.

### Protection of VD Against Degradation Induced by Low pH

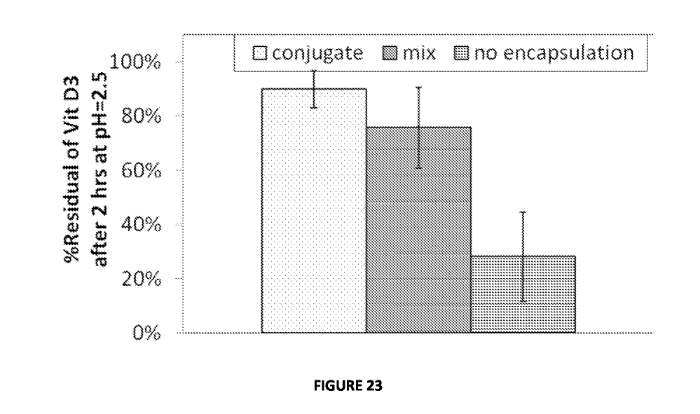

VD degrades at low pH, hence the protection conferred by the nanoencapsulation against its degradation at pH 2.5 was evaluated. The results are presented in FIG. 23. Evidently, the nanoencapsulation protected the VD against degradation due to the low pH, and the conjugates were more effective than the mixture at achieving this protection (although the difference was not statistically significant). Without wishing to be bound by any particular mechanism or theory, it is contemplated that the mechanisms of protection include the immobilization of the vitamin (which reduces its chemical activity), and possibly also the buffering capacity of the protein, which seems to be effective locally, in the vicinity of the protein, thus providing protection to the bound VD.

### Protection Conferred to VD2 by Conjugates During Shelf Life

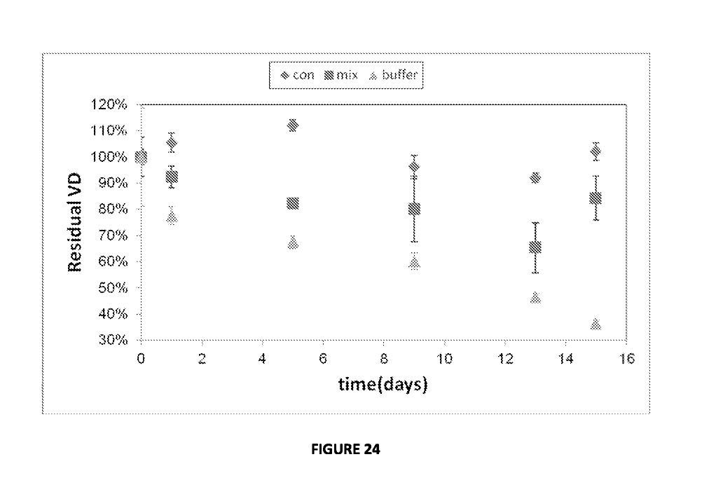

Residual VD during a simulated shelf life study is shown in FIG. 24. The figure clearly shows that the conjugates conferred significantly improved protection against degradation compared to the mixture and the buffer. In addition, the mixture solution conferred significantly better protection to VD than the buffer control.

Without wishing to be bound by any particular mechanism or theory, several possible reasons for this improved protection are contemplated: 1. The interactions between the protein and VD cause its immobilization, thus reducing its reactivity in various reactions. 2. The conjugate nanocapsules shield the entrapped VD from external degradation factors, both chemical (oxidizing agents) and physical (e.g. UV radiation). 3. It was reported that Maillard products act as antioxidants. It is very likely that a combination of these mechanisms confers the observed protection.

### Protection Against Degradation of a Water-Soluble Nutraceutical—EGCG

Epigallocatechin gallate (EGCG) is a water soluble nutraceutical, extracted from green tea. It tends to oxidize at neutral pH and degrade into yellow products that absorbs at 425 nm. It was shown in a previous study that the interaction with milk proteins (beta lactoglobulin), particularly after they undergo heat treatment, can reduce the oxidation rate of EGCG significantly.

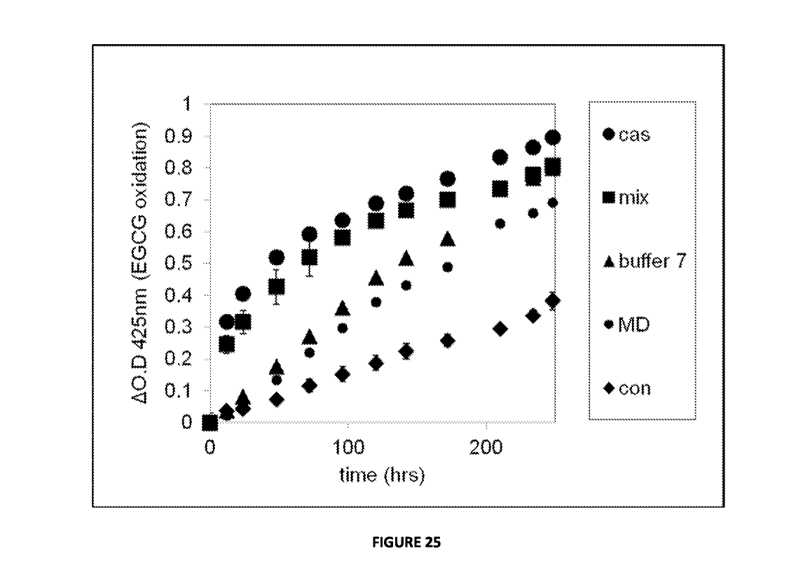

FIG. 25 shows the formation of oxidized EGCG as a function of time in conjugate, MD, CN, and PBS buffer, at pH=7. The figure shows that the conjugate solution conferred significantly better protection against EGCG oxidization at pH=7 compared to all the other alternative systems. In this case, the protein alone and the mixture conferred no protection. This result supports the hypothesis that the conjugate acts as an antioxidant.

Studying Entrapment and Release from the Nanocapsules Upon Simulated Gastric Digestion, by Using Nile Red—a Fluorescent Model for a Hydrophobic Nutraceutical.
Interactions of Nile Red (NR) with Proteins and Solvents

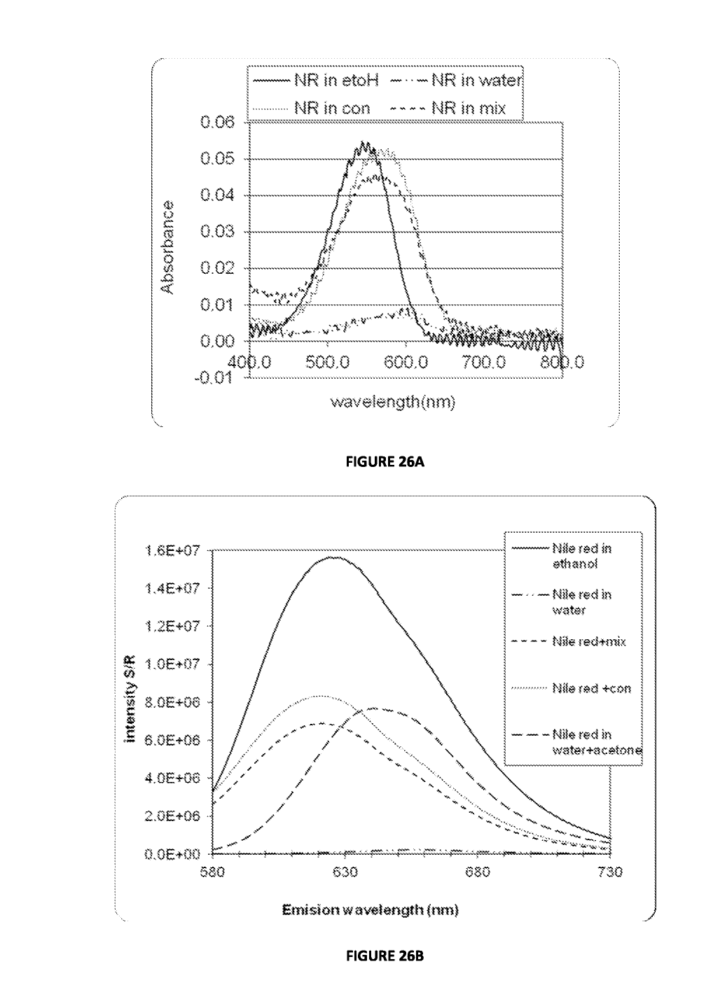

Interaction of the conjugate and mixture with a hydrophobic nutraceutical was examined with a model molecule-NR whose fluorescence emission wavelength and quantum yield depend on the polarity of its environment. NR can be used as a model for Vitamin D2 and Vitamin D3 because all these molecules are very similar in Mw and hydrophobicity (as may be described by log P: log of the octanol-water partition coefficient, P). NR absorbance spectra in ethanol, in the conjugate, in the mixture and in a buffer solution are shown in FIG. 26A. The figure shows that NR's extinction coefficient is much larger in ethanol than in water. According to Sackett et al when in water, NR tends to rapidly adsorb to the glass and leave the solution unless there is a hydrophobic region that it can interact with. In the mixture and conjugate solutions its optical density (OD) is similar to its OD in ethanol, meaning that the NR is bound to the hydrophobic regions of the protein.

Subsequent experiments used 570 nm as the excitation for NR fluorescence measurements, as it is a wavelength that NR absorbs both in water and in conjugate/mix solution. Emission spectra of NR in different solvents (ex:570 nm) are shown in FIG. 26B. The emission intensity of NR in water was about two orders of magnitude lower than its emission in the more hydrophobic solvents like ethanol or acetone:water (50:50 vol) solution. NR emission spectra in conjugate/mix solution showed similar intensity as that in acetone: water, but the peak shifted to longer wavelengths relatively to its emission in acetone: water. This means that when NR is in conjugate or mixture solution it is surrounded by a less polar environment than when it is in an acetone:water 1:1 solution.

In FIG. 26B, the maximum emission intensity of NR in the conjugate seemed higher than its emission in the mixture. Hence the maximum intensity of NR in conjugate and in mixture solutions of different concentrations was examined, in order to validate that trend.

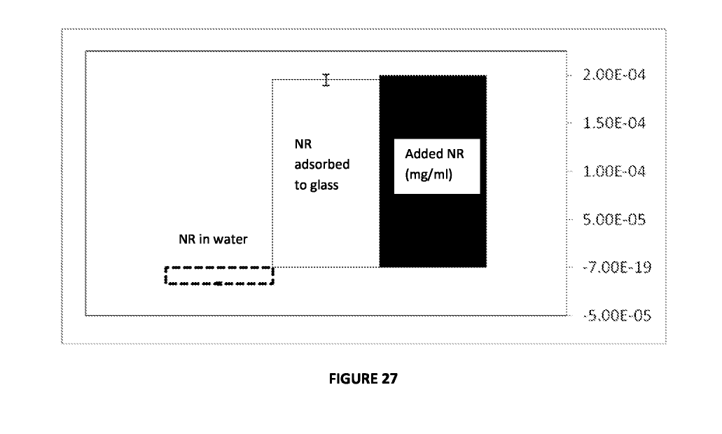

FIG. 27 shows the emission intensity of NR in solutions of mixture and conjugate at different concentrations. It appears that there was no significant difference in NR emission intensities between conjugate and mixture solutions. This phenomenon can be inferred as there is no significant difference between conjugate and mixture loading capacities of hydrophobic molecules similar to NR. This means that caseins' ability to bind to hydrophobic molecules was not decreased due to conjugation with the more hydrophilic MD19.

Adsorption of NR to the Glass Surface when Added to Water: A Way of Probing the Release of a Model Hydrophobic Nutraceutical from Nanoparticles.

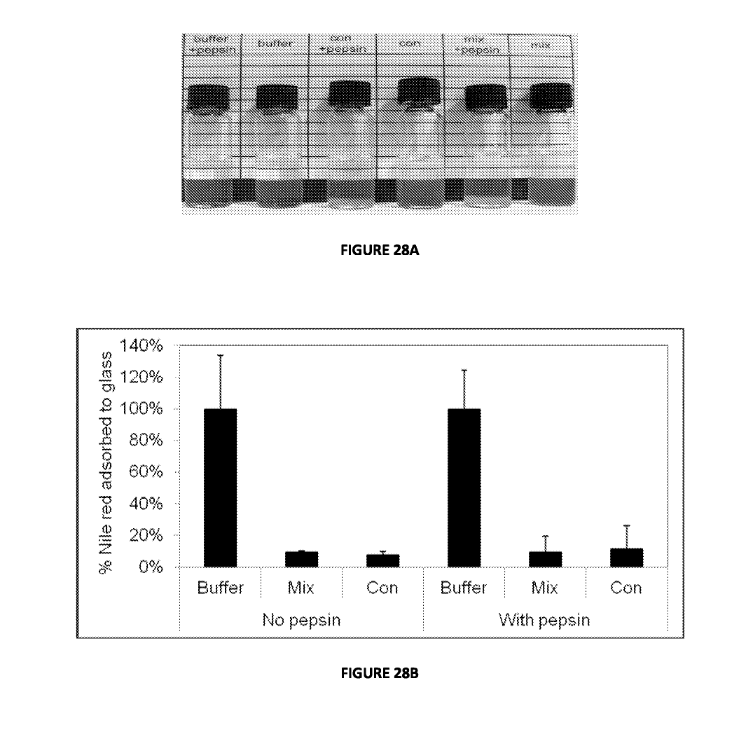

FIG. 28 shows the concentrations of NR adsorbed to glass and of NR in water, compared to the initially added concentration, 2 hours after addition of NR to water. The figure shows that practically all of the NR added to water was adsorbed to the glass (the apparently negative NR concentration in water was an artifact due to subtraction of an extreme value calculated from a linear calibration curve (R²=0.95). The actual concentration should be zero or undetectable.)

### Simulated Gastric Digestion Studies Using NR as a Model for a Hydrophobic Nutraceutical.

NR was used as a model for HN (e.g. VD), to study its release from the conjugate or the mixture during gastric digestion. NR was added to buffer, mixture and conjugate solutions (3 mg/ml CN) at pH=2.5, then pepsin was added to some of the solutions (1:20 pepsin:CN mass ratio). The solutions were stirred for 2 hrs in a 37° C. water bath. After stirring was stopped, aggregates of digested conjugate and mixture began to sediment in the samples containing pepsin.

FIG. 29A shows a photograph of the vials after the 2 hrs incubation. As shown, no color at all is seen in the buffer vial. NR in water absorbs much less light than when in a non-polar environment. It can be seen that in both the mixture and conjugate that were incubated with pepsin, an aggregation followed by sedimentation occurred, as is known to occur during casein digestion by pepsin.

Quantitative results were obtained by transferring the solution and sediment to new vials and addition of water:acetone (1:1). One hour later, water: acetone which extracted NR back from the glass was examined for fluorescence emission. Fluorescence intensity of NR adsorbed to glass is shown in FIG. 29B. The results show that NR was bound to casein both in the mix and in the conjugate solutions at the same high ratio. This result means that the encapsulation efficiency of this model hydrophobic compound under these conditions was about 90% in both the conjugate and the mix. Furthermore, digestion with pepsin did not change the binding of NR to casein peptides. The implication of this phenomenon regarding vitamin D bioavailability from the conjugates should still be examined since vitamin D is naturally being incorporated into chylomicrons. However in a recent study it was found that VD encapsulated in casein micelles was highly bioavailable.

### Conclusions

### Example 3

1. CN-MD Maillard conjugate based nanovehicles for enrichment of HN, having diameters of less than 100 nm, were successfully formed.

2. The complexes of VD-conjugate were less turbid than the ones formed by VD-mixture, and much less turbid than VD dispersed in buffer only, at the high concentrations studied, simulating soft drink concentrates (Completely clear solutions were obtained with nanoencapsulated VD at doses typical for the final drinks).

3. The supernatant conjugate can be used for enrichment of clear beverages even at pH close to 4.6 which is the pI of the native casein, where unconjugated casein nanocapsules would precipitate.

3. Conjugation significantly improved the protection against oxidation conferred to both VD and EGCG.

4. Conjugation does not significantly change the ability of caseins to bind HN.

5. Enzymatic hydrolysis by gastric pepsin of the casein was not followed by release of the hydrophobic molecules from the protein. It is likely that it remains bound to shorter peptides.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the spirit of the invention.

### REFERENCES

1. Livney, Y. D., Complexes and conjugates of biopolymers for delivery of bioactive ingredients via food. In Delivery and controlled release of bioactives in foods and nutraceuticals, 1 ed.; Garti, N., Ed. Woodhead Publishing Ltd.: Cambridge, England, 200; 8 pp 234-250.
2. Dickinson, E. Hydrocolloids at interfaces and the influence on the properties of dispersed systems Food Hydrocolloids 2003, 17, (1), 25-39.
3. Selinheimo, E.; Lampila, P.; Mattinen, M. L.; Buchert, J. Formation of protein—Oligosaccharide conjugates by laccase and tyrosinase Journal of Agricultural and Food Chemistry 2008, 56, (9), 3118-3128.
4. Hattori, M.; Yang, W. H.; Takahashi, K. Functional-Changes of Carboxymethyl Potato Starch by Conjugation with Whey Proteins Journal of Agricultural and Food Chemistry. 1995, 43, (8), 2007-2011.
5. Zaleska, H.; Ring, S.; Tomasik, P. Electrosynthesis of potato starch-casein complexes International Journal of Food Science and Technology 2001, 36, (5), 509-515.
6. Zaleska, H.; Tomasik, P.; Lii, C. Y. Formation of carboxymethyl cellulose-casein complexes by electrosynthesis Food Hydrocolloids 2002, 16, (3), 215-224.
7. Akhtar, M.; Dickinson, E. Emulsifying properties of whey protein-dextran conjugates at low pH and different salt concentrations Colloids and Surfaces, B: Biointerfaces 2003, 31, (1-4), 125-132.
8. Einhorn-Stoll, U.; Ulbrich, M.; Sever, S.; Kunzek, H. Formation of milk protein-pectin conjugates with improved emulsifying properties by controlled dry heating Food Hydrocolloids 2005, 19, (2), 329.340-
9. Neirynck, N.; Van der Meeren, P.; Lukaszewicz-Lausecker, M.; Cocquyt, J.; Verbeken, D.; Dewettinck, K. Influence of pH and biopolymer ratio on whey protein-pectin interactions in aqueous solutions and in O/W emulsions Colloids and Surfaces A—Physicochemical and Engineering Aspects 2007, 298, (1-2), 99-107.
10. Hattori, M.; Numamoto, K.; Kobayashi, K.; Takahashi, K. Functional changes in beta-lactoglobulin by conjugation with cationic saccharides J Agric Food Chem 2000, 48, (6), 2050-2056.
11. Aminlari, M.; Ramezani, R.; Jadidi, F. Effect of Maillard-based conjugation with dextran on the functional properties of lysozyme and casein Journal of the Science of Food and Agriculture 2005, 85, (15), 2617-2624.
12. Morris, G. A.; Sims, I. M.; Robertson, A. J; Furneaux, R. H. Investigation into the physical and chemical properties of sodium caseinate-maltodextrin glyco-conjugates Food Hydrocolloids 2004, 18, (6), 1007-1014.
13. Fechner, A.; Knoth, A.; Scherze, I.; Muschiolik, G. Stability and release properties of double-emulsions stabilised by caseinate-dextran conjugates Food Hydrocolloids 2007, 21, (5-6), 943-952.
14. Guan, J. J.; Qiu, A. Y.; Liu, X. Y.; Hua, Y. F.; Ma, Y. H. Microwave improvement of soy protein isolate-saccharide graft reactions Food Chemistry 2006, 97, (4), 577-585.
15. Diftis, N.; Kiosseoglou, V. Improvement of emulsifying properties of soybean protein isolate by conjugation with carboxymethyl cellulose Food Chemistry 2003, 81, (1), 1-6.
16. Qi, J. R.; Yang, X. Q.; Liao, J. S. Improvement of functional properties of acid-precipitated soy protein by the attachment of dextran through Maillard reaction International Journal of Food Science and Technology 2009, 44, (11), 2296-2302.
17. Fan, J. F.; Zhang, Y. Y.; Szesze, T.; Li, F. J.; Zhou, M. Y; Saito, M.; Tatsumi, E.; Li, L. T. Improving functional properties of soy protein hydrolysate by conjugation with curdlan Journal of Food Science 2006, 71, (5), C285-C291.
18. Kato, A.; Shimokawa, K.; Kobayashi, K. Improvement of the Functional-Properties of Insoluble Gluten by Pronase Digestion Followed by Dextran Conjugation Journal of Agricultural and Food Chemistry 1991, 39, (6), 1053-1056.
19. Hiller, B.; Lorenzen, P. C. Functional properties of milk proteins as affected by Maillard reaction induced oligomerisation Food Research International 2010, 43, (4), 1155-1166.
20. Kato, A. Industrial applications of Maillard-type protein-polysaccharide conjugates Food Science and Technology Research 2002, 8, (3), 193-199.
21. Kim, J. S.; Lee, Y. S. Antioxidant activity of Maillard reaction products derived from aqueous glucose/glycine, diglycine, and triglycine model systems as a function of heating time Food Chemistry 2009, 116, (1), 227-232.
22. Yilmaz, Y.; Toledo, R. Antioxidant activity of water-soluble Maillard reaction products Food Chemistry 2005, 93, (2), 273-278.
23. Mu, M. F.; Pan, X. Y.; Yao, P.; Jiang, M. Acidic solution properties of beta-casein-graft-dextran copolymer prepared through Maillard reaction Journal of Colloid and Interface Science 2006, 301, (1), 98-106.
24. Pan, X.; Mu, M.; Hu, B.; Yao, P.; Jiang, M. Micellization of casein-graft-dextran copolymer prepared through Maillard reaction Biopolymers 2006, 81, (1), 29-38.
25. Pan, X. Y.; Yao, R; Jiang, M. Simultaneous nanoparticle formation and encapsulation driven by hydrophobic interaction of casein-graft-dextran and beta-carotene Journal of Colloid and Interface Science 2007, 315, (2), 456-463.
26. Choi, K. O.; Ryu, J.; Kwak, H. S.; Ko, S. Spray-dried Conjugated Linoleic Acid Encapsulated with Maillard Reaction Products of Whey Proteins and Maltodextrin Food Science and Biotechnology 2010, 19, (4), 957-965.
27. J. Li, P. Yao., Self-Assembly of Ibuprofen and Bovine Serum Albumin-Dextran Conjugates Leading to Effective Loading of the Drug. Langmuir, 2009. 25(11): p. 6385-6391.
28. Wooster, T. J. and M. A. Augustin, The emulsion flocculation stability of protein-carbohydrate diblock copolymers. Journal of Colloid and Interface Science, 2007. 313(2): p. 665-675.
29. Hiller, B. and P. C. Lorenzen, Surface hydrophobicity of physicochemically and enzymatically treated milk proteins in relation to techno-functional properties. Journal of Agricultural and Food Chemistry, 2008. 56(2): p. 461-468.
30. Serfert, Y., S. Drusch, and K. Schwarz, Chemical stabilisation of oils rich in long-chain polyunsaturated fatty acids during homogenisation, microencapsulation and storage. Food Chemistry, 2009. 113(4): p. 1106-1112.
31. Nursten, H., The Maillard Reaction Chemistry, Biochemistry and Implications. 2005, Reading: The Royal Society of Chemistry.
32. O'Regan, J. and D. M. Mulvihill, Sodium caseinate-maltodextrin conjugate hydrolysates: Preparation, characterisation and some functional properties. Food Chemistry, 2010. 123(1): p. 21-31.
33. Goodno, C. C.; Swaisgood, H. E.; Catignani, G. L. A Fluorimetric Assay for Available Lysine in Proteins Analytical Biochemistry 1981, 115, (1), 203-211.
34. Vigo, M. S.; Malec, L. S.; Gomez, R. G.; Llosa, R. A. Spectrophotometric Assay Using Ortho-Phthaldialdehyde for Determination of Reactive Lysine in Dairy-Products Food Chemistry 1992, 44, (5), 363-365.
35. Shapira, A.; Assaraf, Y. G.; Livney, Y. D. Beta-Casein Nano-Vehicles for Oral Delivery of Chemotherapeutic Drugs Nanomedicine: Nanotechnology, Biology, and Medicine 2010, 6, 119-126.
36. Semo, E.; Kesselman, E.; Damino, D.; Livney, Y. D. Casein micelle as a natural nano-capsular vehicle for nutraceuticals Food Hydrocolloids 2007, 21, (5-6), 936-942.
37. Kazmi, S. A.; Vieth, R.; Rousseau, D. Vitamin D3 fortification and quantification in processed dairy products International Dairy Journal 2007, 17, (7), 753-759.

We claim:

1. A composition for enrichment of beverages, comprising a nutraceutical encapsulated or entrapped or protected by a conjugate, the conjugate comprising a protein or peptide covalently bonded to a polysaccharide or oligosaccharide, wherein the particle size of said composition is sufficiently small such that, when added to a beverage, a clear solution is formed.

2. The composition according to claim 1, wherein the clear solution has an absorbance at 600 nm of less than about 0.1.

3. The composition according to claim 1, wherein the conjugate is formed by a Maillard reaction or a Maillard-type reaction.

4. The composition according to claim 1, wherein the conjugate comprises a Schiff base, an Amadori rearrangement product, or keto-enol tautomers.

5. The composition according to claim 1, wherein the average particle diameter of said composition is between about 50 and 100 nm.

6. The composition according to claim 1, wherein the average particle diameter of said composition is less than about 50 nm.

7. The composition according to claim 1, wherein the nutraceutical is a hydrophobic poorly water-soluble or water insoluble nutraceutical.

8. The composition according to claim 7, wherein the solubility of the nutraceutical is below about 30 mg/ml in water.

9. The composition according to claim 1, wherein the nutraceutical is a hydrophilic or water soluble nutraceutical.

10. The composition according to claim 9, wherein the solubility of the nutraceutical is about or above 30 mg/ml in water.

11. The composition according to claim 1, wherein the nutraceutical is an amphiphilic nutraceutical.

12. The composition according to claim 1, wherein the nutraceutical is a fat-soluble vitamin selected from vitamin A, vitamin E, vitamin D and vitamin K, and derivatives thereof.

13. The composition according to claim 1, wherein the nutraceutical is an unsaturated fatty acid.

14. The composition according to claim 13, wherein the unsaturated fatty acid is selected from the group consisting of linoleic acid, conjugated linoleic acid (CLA), omega-3 fatty acids including alpha linolenic acid, DHA and EPA and their esters including glycerol esters.

15. The composition according to claim 1, wherein the nutraceutical is selected from a phytochemical, a phytosterol, a polyphenol, a tannin, a catechin, a flavonoid, an isoflavone, an isoflavonoid, a neoflavonoid, a lignin, a coumestan, and a stilbene.

16. The composition according to claim 15, wherein the nutraceutical is a selected from the group consisting of epigallocatechin gallate (EGCG), epicatechin (EC), epicatechin gallate (ECG), epigallocatechin (EGC), punicalagin, .beta.-sitosterol, campesterol, stigmasterol, genistein, daidzein, resveratrol, trans-resveratrol, matairesinol, coumestrol, curcumin and coenzyme-Q10.

17. The composition according to claim 1, wherein the nutraceutical is a sterol cholesterol or a derivative thereof, or wherein the nutraceutical is selected from the group consisting of .alpha.-carotene, .beta.-carotene, .gamma.-carotene, lycopene, lutein, zeaxanthin, and astaxanthin.

18. The composition according to claim 1, wherein the protein in said conjugate is a vegetable protein, an animal protein, a milk protein, an egg protein, a fungi protein, a microbial protein, an algae protein or any hydrolyzate, peptide or combination thereof.

19. (canceled)

20. (canceled)

21. (canceled)

22. (canceled)

23. (canceled)

24. (canceled)

25. (canceled)

26. A method for the enrichment of beverages with at least one nutraceutical, the method comprising the step of adding the composition according to claim 1 to a beverage.

27. A method for the enrichment of beverages with at least one nutraceutical, the method comprising the step of adding to a beverage a nutraceutical encapsulated, or entrapped or protected by a conjugate, the conjugate comprising a protein or peptide covalently bonded to a polysaccharide or oligosaccharide, wherein the particle size of said composition is sufficiently small such that, when added to said beverage, a clear solution is formed

28. (canceled)

29. (canceled)

30. (canceled)

31. (canceled)

32. (canceled)

33. (canceled)
